(19) United States
(12) Patent Application Publication
(10) Pub. No.: US 2011/0229942 A1
(43) Pub. Date: Sep. 22, 2011

(54) Microbial Conversion of Oils and Fatty Acids to High-Value Chemicals

(75) Inventors: Paul Campbell, Houston, TX (US); Ramon Gonzalez, Pearland, TX (US)

(73) Assignee: Glycos Biotechnologies, Incorporated, Houston, TX (US)

(21) Appl. No.: 12/747,436

(22) PCT Filed: Dec. 15, 2008
(86) PCT No.: PCT/US08/13707

§ 371 Date: May 18, 2011

Related U.S. Application Data
(60) Provisional application No. 61/189,427, filed on Aug. 19, 2008.
(60) Provisional application No. 61/007,481, filed on Dec. 13, 2007.

(51) Int. Cl. C12P 17/04; C12N 1/21; C12N 1/15; C12N 1/19; C12P 7/06; C12P 7/54; C12P 7/46; C12P 7/18; C12P 7/52; C12P 7/28; C12P 7/62; C12P 7/04; C12P 7/16; C12P 7/42; C12P 13/00

(52) U.S. Cl. 435/126; 435/252.3; 435/254.11; 435/252.31; 435/252.33; 435/254.21; 435/254.23; 435/254.3; 435/254.4; 435/254.2; 435/161; 435/140; 435/145; 435/158; 435/141; 435/150; 435/135; 435/157; 435/160; 435/137; 435/128

(57) ABSTRACT

Microorganisms for the production of high-value chemicals from free fatty acids are provided. The microorganisms comprise genetic mutations that alter fatty acid metabolism. The genetic mutations include a mutation or deletion of a fadR gene in which the FadR enzyme activity is partially or substantially eliminated and a mutation in an atoC gene that provides overexpression of the microorganism's ato operon. Methods of using the microorganisms to produce high-value chemicals are also provided. The high-value chemicals include ethanol, methyl acetate, succinate, gamma-butyrolactone, 1,4-butanediol, acetone, iso-propanol, butyrate, butanol, mevalonate, propionate, ethanolamine and 1,2-propanediol.

### CROSS-REFERENCE TO RELATED APPLICATIONS

This application claims the benefit of U.S. Provisional Patent Application Ser. Nos. 61/007,481, filed on Dec. 13, 2007, and 61/189,427, filed on Aug. 19, 2008, both of which are incorporated herein by reference in their entirety.

### BACKGROUND OF THE INVENTION

The present disclosure generally relates to the metabolic engineering of microorganisms to produce high-value chemicals.

Currently, many high-value chemicals or fuels are typically produced by chemical synthesis from hydrocarbons, including petroleum oil and natural gas. However, as the concerns of energy security, increasing oil and natural gas prices, and global warming escalate, industry is seeking ways to replace chemicals made from non-renewable feedstocks by harsh processes with chemicals made from renewable feedstocks with environmentally friendly processes. In the transportation fuel sector, this trend has led to the growth of the bioethanol and biodiesel industries.

Although ethanol can be produced from hydrocarbons using a traditional chemical synthesis, using microorganisms to produce ethanol by fermentation of a sugar feedstock is an alternative approach. While this approach does not have the problems mentioned above, there are still drawbacks. For example, the maximum theoretical yield (weight basis) of ethanol from a glucose or xylose feedstock for bacterial fermentation is only 0.51, as the required chemical reactions result in a shortage of reducing equivalents. In addition, the process creates a considerable amount of carbon dioxide (0.49 theoretical yield), one of the primary greenhouse gases implicated in global warming.

Therefore, there remains a need for microorganisms that are capable of producing higher yields of ethanol and other high-value chemicals from a renewable carbon source with reduced amounts of carbon dioxide released into the atmosphere.

### SUMMARY OF THE INVENTION

Embodiments of the present disclosure generally provide microorganisms and methods of using the microorganisms for the production of high value chemicals. The microorganisms comprise reduced fadR expression or function, or increased expression of the fad regulon, and increased expression of the atoDAEB genes, for example through mutation in an atoC gene that provides overexpression of the microorganism's ato operon.

Embodiments of the present disclosure also provide methods of producing a desired product using a microorganism. The microorganism may be any of the microorganisms provided according to embodiments of the disclosure. The methods generally comprise providing a medium comprising free fatty acids, monoglycerides, diglycerides, triglycerides, phospholipids, or a combination thereof and culturing the microorganism in the medium under conditions such that the microorganism converts the free fatty acids, monoglycerides, diglycerides, triglycerides, phospholipids, or combinations thereof, into the desired product. The desired product may be a high-value chemical such as ethanol, acetate, succinate, gamma-butyrolactone, 1,4-butanediol, methyl acetate, acetone, isopropanol, butyrate, butanol, mevalonate, ethanolamine, propionate, or 1,2-propanediol.

In one embodiment, the microorganism further comprises a gene expressing an alcohol dehydrogenase that is active under aerobic conditions, and the high value chemical produced by the microorganism is ethanol. Thus, the present disclosure provides a microorganism comprising reduced fadR expression or function, or increased expression of the fad regulon, increased expression of the atoDAEB genes, and a nucleic acid sequence encoding an alcohol dehydrogenase protein that is active under aerobic conditions. In certain embodiments, the microorganism is a bacterium, for example a member of the genus Escherichia, Lactobacillus, Lactococcus, Bacillus, Paenibacillus, Klebsiella, Citrobacter, Clostridium, or Zymomonas. In certain embodiments, the microorganism is Escherichia coli. In other embodiments, the microorganism is a fungus, for example a member of the genus Saccharomyces, Pichia, Schizosaccharomyces, Aspergillus, or Neurospora. In particular embodiments, the microorganism is Saccharomyces cerevisiae, Pichia pastoris, or Schizosaccharomyces pombe.

In certain embodiments the microorganism comprises reduced fadR expression, for example by a mutation in the fadR gene promoter, including, but not limited to, a point mutation, deletion mutation, or insertion mutation in the fadR gene promoter. In other embodiments, the microorganism comprises reduced fadR function, for example by a mutation in the fadR gene, including, but not limited to, a point mutation, deletion mutation, or insertion mutation in the fadR gene. In still other embodiments, the microorganism comprises increased expression of the fad regulon, for example increased expression of the fadL, fadD, fadE, fadBA, and fadH genes. In certain aspects, expression of the fadL, fadD, fadE, fadBA, and fadH genes are increased to about the same level, for example by operably linking the fadL, fadD, fadE, fadBA, and fadH genes to the same promoter, while in other aspects expression of the fadL, fadD, fadE, fadBA, and fadH genes are increased to different levels, for example by operably linking the fadL, fadD, fadE, fadBA, and fadH genes to two or more different promoters. In further embodiments, since CoA thioesters bind to and change the conformation of fadR leading to derepression of the fad genes, fadR function is reduced by providing the microorganism with at least a first CoA thioester.

In certain aspects, the microorganism comprises increased expression of the atoDAEB genes, which can occur in a number of different ways, including, but not limited to, by increased expression of the ato operon, for example by comprising a nucleic acid sequence encoding an atoC protein comprising a constitutive mutation, or by comprising a nucleic acid sequence encoding the ato operon operably linked to a constitutive or inducible promoter, or by comprising a first nucleic acid sequence encoding the atoD gene operably linked to a first constitutive or inducible promoter, a second nucleic acid sequence encoding the atoA gene operably linked to a second constitutive or inducible promoter, a third nucleic acid sequence encoding the atoE gene operably linked to a third constitutive or inducible promoter, and a fourth nucleic acid sequence encoding the atoB gene operably linked to a fourth constitutive or inducible promoter. In certain embodiments, the first, second, third, and fourth constitutive or inducible promoter are the same constitutive or inducible promoter, while in other embodiments the first, second, third, and fourth constitutive or inducible promoter are different constitutive or inducible promoters.

In certain embodiments, the microorganism comprises a nucleic acid sequence encoding a mutant alcohol dehydrogenase protein that is active under aerobic conditions. In particular embodiments, the microorganism comprises a nucleic acid sequence encoding an E. coli alcohol dehydrogenase protein comprising a non-acidic amino acid residue at position 568 of the amino acid sequence, for example a basic amino acid residue at position 568 of the amino acid sequence. In certain aspects, the microorganism comprises a nucleic acid sequence encoding an E. coli alcohol dehydrogenase protein comprising a lysine residue at position 568 of the amino acid sequence. In other aspects, the microorganism comprises a nucleic acid sequence encoding a Saccharomyces cerevisiae alcohol dehydrogenase protein that is active under aerobic conditions.

In certain embodiments, the microorganism further comprises reduced NADH dehydrogenase expression or activity, for example a deletion of the NADH dehydrogenase gene. In other aspects, the microorganism further comprises a nucleic acid sequence encoding a fadE protein that uses NAD+ or NADP+ as a co-factor. Such a fadE protein can be, for example, a mutant fadE protein that uses NAD+ or NADP+ as a co-factor, or a fadE protein that naturally uses NAD+ or NADP+ as a co-factor, such as a fadE protein from Mycobacterium smegmatis or Euglena gracilis.

Therefore, the disclosure provides a method of converting free fatty acids to ethanol, comprising culturing a microorganism comprising reduced fadR expression or function, or increased expression of the fad regulon, increased expression of the atoDAEB genes, and a nucleic acid sequence encoding an alcohol dehydrogenase protein that is active under aerobic conditions, in a culture medium comprising free fatty acids, monoglycerides, triglycerides, phospholipids, or a combination thereof, thereby converting said free fatty acids to ethanol.

In certain embodiments, the concentration of free fatty acids, monoglycerides, diglycerides, triglycerides, phospholipids, or combination thereof, is about 0.1% to about 10%, or about 2% to about 5%. In particular embodiments, the concentration of free fatty acids, monoglycerides, diglycerides, triglycerides, phospholipids, or combination thereof, in the culture medium is maintained at about 0.1% to about 10% during the culturing of said microorganism. In some aspects, the culture medium further comprises a nitrate or nitrite salt.

The present disclosure thus provides a method of producing ethanol, comprising culturing a microorganism comprising reduced fadR expression or function, or increased expression of the fad regulon, increased expression of the atoDAEB genes, and a nucleic acid sequence encoding an alcohol dehydrogenase protein that is active under aerobic conditions, in a culture medium comprising free fatty acids, monoglycerides, diglycerides, triglycerides, phospholipids, or a combination thereof, thereby producing ethanol. Recovery of ethanol can be accomplished by standard techniques known to those of skill in the art, such as recovery from the fermentation broth by distillation, pervaporation, or liquid-liquid extraction.

In other embodiments, the microorganism further comprises genes overexpressing an acetate kinase enzyme and a phosphate acetyltransferase enzyme or genes overexpressing an ADP-forming acetyl-CoA synthetase, and the high value chemical produced by the microorganism is acetate. The present disclosure therefore provides a microorganism comprising reduced fadR expression or function, or increased expression of the fad regulon, increased expression of the atoDAEB genes, and increased expression of an acetate kinase gene or a phosphate acetyltransferase gene, increased expression of an ADP-forming acetyl-CoA synthetase activity, or expression of an acetyl-CoA synthetase activity that functions primarily to produce acetate from acetyl-CoA. In certain embodiments, the microorganism comprises increased expression of an acetate kinase gene and a phosphate acetyltransferase gene, for example by increased expression of an ackA-pta operon. In other embodiments, the microorganism comprises increased expression of an ADP-forming acetyl-CoA synthetase activity, for example by increased expression of the acdA and acdB genes of Pyrococcus furiosus. In yet other embodiments, the microorganism comprises expression of an acetyl-CoA synthetase activity that functions primarily to produce acetate from acetyl-CoA. In certain aspects, the microorganism comprises an endogenous acetyl-CoA synthetase activity that functions primarily to produce acetate from acetyl-CoA, for example the acs gene of E. coli, while in other aspects, the microorganism comprises a mutant acetyl-CoA synthetase activity that functions primarily to produce acetate from acetyl-CoA, and in further aspects, the microorganism comprises an exogenous acetyl-CoA synthetase activity that functions primarily to produce acetate from acetyl-CoA.

Thus, the present disclosure provides a method of converting fatty acids to acetate, comprising culturing a microorganism comprising reduced fadR expression or function, or increased expression of the fad regulon, increased expression of the atoDAEB genes, and increased expression of an acetate kinase gene or a phosphate acetyltransferase gene, increased expression of an ADP-forming acetyl-CoA synthetase activity, or expression of an acetyl-CoA synthetase activity that functions primarily to produce acetate from acetyl-CoA, in a culture medium comprising free fatty acids, monoglycerides, triglycerides, phospholipids, or a combination thereof, thereby converting said free fatty acids to acetate.

The present disclosure also provides a method of producing acetate, comprising culturing a microorganism comprising reduced fadR expression or function, or increased expression of the fad regulon, increased expression of the atoDAEB genes, and increased expression of an acetate kinase gene or a phosphate acetyltransferase gene, increased expression of an ADP-forming acetyl-CoA synthetase activity, or expression of an acetyl-CoA synthetase activity that functions primarily to produce acetate from acetyl-CoA, in a culture medium comprising free fatty acids, monoglycerides, diglycerides, triglycerides, phospholipids, or a combination thereof, thereby producing acetate. Recovery of acetate can be accomplished by standard techniques known to those of skill in the art.

In another embodiment, the microorganism further comprises a knocked-out sucA gene or sucB gene, or both, and a knocked-out sdhA gene or sdhB gene, or both. The high value chemical produced by the microorganism is succinate. Thus, the present disclosure provides a microorganism comprising reduced fadR expression or function, or increased expression of the fad regulon, increased expression of the atoDAEB genes, reduced sucA or sucB expression or function, and reduced sdhA or sdhB expression or function. In certain embodiments, the microorganism comprises reduced sucA and sucB expression or function or reduced sdhA and sdhB expression or function, while in other embodiments, the microorganism comprises reduced sucA and sucB expression or function, and reduced sdhA and sdhB expression or function. In particular aspects, the microorganism further comprises reduced iclR expression or function or increased expression of an aceA and an aceB gene.

The present disclosure thus provides a method of converting fatty acids to succinate, comprising culturing a microorganism comprising reduced fadR expression or function, or increased expression of the fad regulon, increased expression of the atoDAEB genes, reduced sucA or sucB expression or function, and reduced sdhA or sdhB expression or function, in a culture medium comprising free fatty acids, monoglycerides, diglycerides, triglycerides, phospholipids, or a combination thereof, thereby converting said free fatty acids to succinate.

The present disclosure also provides a method of producing succinate, comprising culturing a microorganism comprising reduced fadR expression or function, or increased expression of the fad regulon, increased expression of the atoDAEB genes, reduced sucA or sucB expression or function, and reduced sdhA or sdhB expression or function, in a culture medium comprising free fatty acids, monoglycerides, diglycerides, triglycerides, phospholipids, or a combination thereof, thereby producing succinate. Recovery of succinate can be accomplished by standard techniques known to those of skill in the art, such as recovery from the fermentation broth by ion exchange, crystallization/precipitation, or liquid-liquid extraction.

In another embodiment, the microorganism further comprises a knocked-out sucA gene or sucB gene, or both, and a knocked-out sdhA gene or sdhB gene, or both, a gene overexpressing a succinic semialdehyde dehydrogenase, a gene overexpressing a gamma-hydroxybutyric acid dehydrogenase, and a gene overexpressing a lactonase. The high value chemical produced by the microorganism is gamma-butyrolactone. The present disclosure thus provides a microorganism comprising reduced fadR expression or function, or increased expression of the fad regulon, increased expression of the atoDAEB genes, reduced sucA or sucB expression or function, reduced sdhA or sdhB expression or function, increased expression or function of one or more of succinic semialdehyde dehydrogenase, succinyl-CoA:CoA transferase, or succinate-CoA ligase, increased gamma-hydroxybutyric acid dehydrogenase expression or function, and increased lactonase expression or function. In certain embodiments, the microorganism comprises increased expression or function of two or more of succinic semialdehyde dehydrogenase, succinyl-CoA:CoA transferase, or succinate-CoA ligase, while in other embodiments, the microorganism comprises increased expression or function of succinic semialdehyde dehydrogenase, succinyl-CoA:CoA transferase, and succinate-CoA ligase.

While the succinic semialdehyde dehydrogenase can be from any source, in certain embodiments, the succinic semialdehyde dehydrogenase is AIdA (NAD+-linked) or Sad (NAD+-dependent) from E. coli, Gabd1 from Mycobacterium tuberculosis, AldH5A1 from Homo sapiens, Ssadh1 from Arabidopsis thaliana, or AttK from Agrobacterium tumefaciens. Likewise, the succinyl-CoA:CoA transferase can be from a variety of sources, but in certain embodiments the succinyl-CoA:CoA transferase is succinyl-CoA:CoA transferase from Clostridium kluyveri, acetyl:Succinate Co-A transferase from Tritrichomonas foetus or Trypanosoma brucei, or acetyl-CoA synthetase (AMP-forming) from E. coli. Similarly, while the succinate-CoA ligase can be from any of a variety of different sources, in certain aspects the succinate-CoA ligase is succinyl-CoA synthetase from E. coli. Additionally, the gamma-hydroxybutyric acid dehydrogenase can be from any number of sources, but in particular embodiments the gamma-hydroxybutyric acid dehydrogenase is 4-hydroxybutyrate dehydrogenase (NADPH-dependent) from Arabidopsis thaliana, NADPH-dependent alcohol dehydrogenase from Bos Taurus, Homo sapiens, Rattus norvegicus, Oryctolagus cuniculus, or Sus scrofa, or BlcB from Agrobacterium tumefaciens. Furthermore, while lactonase from any source can be used, in certain embodiments the lactonase is lactonase from Homo sapiens or Mus musculus, AttM from Agrobacterium tumefaciens, or lipase B48 from Candida antarctica.

In particular embodiments, the microorganism further comprises reduced NADH dehydrogenase expression or activity, for example by deletion of all or a portion of the NADH dehydrogenase gene.

Therefore, the present disclosure provides a method of converting fatty acids to gamma-butyrolactone, comprising culturing a microorganism comprising reduced fadR expression or function, or increased expression of the fad regulon, increased expression of the atoDAEB genes, reduced sucA or sucB expression or function, reduced sdhA or sdhB expression or function, increased expression or function of one or more of succinic semialdehyde dehydrogenase, succinyl-CoA:CoA transferase, or succinate-CoA ligase, increased gamma-hydroxybutyric acid dehydrogenase expression or function, and increased lactonase expression or function, in a culture medium comprising free fatty acids, monoglycerides, triglycerides, phospholipids, or a combination thereof, thereby converting said free fatty acids to gamma-butyrolactone.

The present disclosure also provides a method of producing gamma-butyrolactone, comprising culturing a microorganism comprising reduced fadR expression or function, or increased expression of the fad regulon, increased expression of the atoDAEB genes, reduced sucA or sucB expression or function, reduced sdhA or sdhB expression or function, increased expression or function of one or more of succinic semialdehyde dehydrogenase, succinyl-CoA:CoA transferase, or succinate-CoA ligase, increased gamma-hydroxybutyric acid dehydrogenase expression or function, and increased lactonase expression or function, in a culture medium comprising free fatty acids, monoglycerides, diglycerides, triglycerides, phospholipids, or a combination thereof, thereby producing gamma-butyrolactone. Recovery of gamma-butyrolactone can be accomplished by standard techniques known to those of skill in the art, including, but not limited to, recovery from the fermentation broth by vacuum distillation or liquid-liquid extraction.

In a further embodiment, the microorganism further comprises a knocked-out sucA gene or sucB gene, or both, and a knocked-out sdhA gene or sdhB gene, or both, a gene overexpressing a succinic semialdehyde dehydrogenase, a gene overexpressing a gamma-hydroxybutyric acid dehydrogenase, and a gene overexpressing an aldehyde dehydrogenase, and a gene overexpressing an alcohol dehydrogenase. The high value chemical produced by the microorganism is 1,4-butanediol. Therefore, the present disclosure provides a microorganism comprising reduced fadR expression or function, or increased expression of the fad regulon, increased expression of the atoDAEB genes reduced sucA or sucB expression or function, reduced sdhA or sdhB expression or function, increased expression or function of one or more of succinic semialdehyde dehydrogenase, succinyl-CoA:CoA transferase, or succinate-CoA ligase, increased gamma-hydroxybutyric acid dehydrogenase expression or function, increased aldehyde dehydrogenase expression or function, and increased alcohol dehydrogenase expression or function. In certain embodiments, the microorganism comprises increased expression or function of two or more of succinic semialdehyde dehydrogenase, succinyl-CoA:CoA transferase, or succinate-CoA ligase, while in other embodiments, the microorganism comprises increased expression or function of succinic semialdehyde dehydrogenase, succinyl-CoA:CoA transferase, and succinate-CoA ligase.

While aldehyde dehydrogenase from any source can be utilized, in certain embodiments the aldehyde dehydrogenase is AldH or YdcW from E. coli, aminobutyraldehyde dehydrogenase from Arthrobacter sp. TMP-1, or KauB from Pseudomonas aeruginosa. Similarly, while any source of alcohol dehydrogenase is suitable for use, in particular embodiments the alcohol dehydrogenase is 1,3-propanediol dehydrogenase from Citrobacter freundi or Klebsiella pneumoniae.

In certain aspects the microorganism further comprises reduced NADH dehydrogenase expression or activity, such as, for example, a deletion of all or a portion of the NADH dehydrogenase gene.

The present disclosure therefore provides a method of converting fatty acids to 1,4-butanediol, comprising culturing a microorganism comprising reduced fadR expression or function, or increased expression of the fad regulon, increased expression of the atoDAEB genes, reduced sucA or sucB expression or function, reduced sdhA or sdhB expression or function, increased expression or function of one or more of succinic semialdehyde dehydrogenase, succinyl-CoA:CoA transferase, or succinate-CoA ligase, increased gamma-hydroxybutyric acid dehydrogenase expression or function, increased aldehyde dehydrogenase expression or function, and increased alcohol dehydrogenase expression or function, in a culture medium comprising free fatty acids, monoglycerides, diglycerides, triglycerides, phospholipids, or a combination thereof, thereby converting said free fatty acids to 1,4-butanediol.

The present disclosure also provides a method of producing 1,4-butanediol, comprising culturing a microorganism comprising reduced fadR expression or function, or increased expression of the fad regulon, increased expression of the atoDAEB genes, reduced sucA or sucB expression or function, reduced sdhA or sdhB expression or function, increased expression or function of one or more of succinic semialdehyde dehydrogenase, succinyl-CoA:CoA transferase, or succinate-CoA ligase, increased gamma-hydroxybutyric acid dehydrogenase expression or function, increased aldehyde dehydrogenase expression or function, and increased alcohol dehydrogenase expression or function, in a culture medium comprising free fatty acids, monoglycerides, diglycerides, triglycerides, phospholipids, or a combination thereof, thereby producing 1,4-butanediol. Recovery of 1,4-butanediol can be accomplished by standard techniques known to those of skill in the art, including, but not limited to, recovery from the fermentation broth by vacuum distillation or liquid-liquid extraction.

In another embodiment, the microorganism further comprises a knocked-out sucA gene or sucB gene, or both, a knocked-out sdhA gene or sdhB gene, or both, a gene overexpressing a methylmalonyl-CoA mutase, a gene overexpressing a methylmalonyl-CoA decarboxylase, and a gene overexpressing a propionyl-CoA:succinate CoA transferase. The high value chemical produced by the microorganism is propionate. The present disclosure thus provides a microorganism comprising reduced fadR expression or function, or increased expression of the fad regulon, increased expression of the atoDAEB genes, reduced sucA or sucB expression or function, reduced sdhA or sdhB expression or function, increased methylmalonyl-CoA mutase expression or function, increased methylmalonyl-CoA decarboxylase expression or function, and increased propionyl-CoA:succinate CoA transferase expression or function. While the methylmalonyl-CoA mutase, methylmalonyl-CoA decarboxylase, and propionyl-CoA:succinate CoA transferase can be from any source, in certain embodiments the methylmalonyl-CoA mutase is a scpA gene, the methylmalonyl-CoA decarboxylase is a scpB gene, and the propionyl-CoA:succinate CoA transferase is a scpC gene.

The present disclosure thus provides a method of converting fatty acids to propionate, comprising culturing a microorganism comprising reduced fadR expression or function, or increased expression of the fad regulon, increased expression of the atoDAEB genes, reduced sucA or sucB expression or function, reduced sdhA or sdhB expression or function, increased methylmalonyl-CoA mutase expression or function, increased methylmalonyl-CoA decarboxylase expression or function, and increased propionyl-CoA:succinate CoA transferase expression or function, in a culture medium comprising free fatty acids, monoglycerides, diglycerides, triglycerides, phospholipids, or a combination thereof, thereby converting said free fatty acids to propionate.

The present disclosure also provides a method of producing propionate, comprising culturing a microorganism comprising reduced fadR expression or function, or increased expression of the fad regulon, increased expression of the atoDAEB genes, reduced sucA or sucB expression or function, reduced sdhA or sdhB expression or function, increased methylmalonyl-CoA mutase expression or function, increased methylmalonyl-CoA decarboxylase expression or function, and increased propionyl-CoA:succinate CoA transferase expression or function, in a culture medium comprising free fatty acids, monoglycerides, diglycerides, triglycerides, phospholipids, or a combination thereof, thereby producing propionate. Recovery of propionate can be accomplished by standard techniques known to those of skill in the art.

In another embodiment, the microorganism further comprises a gene overexpressing an acetyl-CoA acetyltransferase, a gene overexpressing an acetyl-CoA:acetoacetyl-CoA transferase, and a gene overexpressing an acetoacetate decarboxylase. The high value chemical produced by the microorganism is acetone. The present disclosure therefore provides a microorganism comprising reduced fadR expression or function, or increased expression of the fad regulon, increased expression of the atoDAEB genes, increased acetyl-CoA acetyltransferase expression or function, increased acetoacetyl-CoA transferase expression or function, and increased acetoacetate decarboxylase expression or function. In certain embodiments, the acetyl-CoA acetyltransferase can be either endogenous or heterologous acetyl-CoA acetyltransferase, and in other embodiments the acetoacetyl-CoA transferase can be endogenous or heterologous acetoacetyl-CoA transferase. In particular embodiments, the acetyl-CoA acetyltransferase is AtoB from E. coli or thiolase from Clostridium acetobutylicum, the acetoacetyl-CoA transferase is AtoAD from E. coli or CtfAB from Clostridium acetobutylicum, and the acetoacetate decarboxylase is Adc from Clostridium acetobutylicum.

The present disclosure thus provides a method of converting fatty acids to acetone, comprising culturing a microorganism comprising reduced fadR expression or function, or increased expression of the fad regulon, increased expression of the atoDAEB genes, increased acetyl-CoA acetyltransferase expression or function, increased acetoacetyl-CoA transferase expression or function, and increased acetoacetate decarboxylase expression or function, in a culture medium comprising free fatty acids, monoglycerides, diglycerides, triglycerides, phospholipids, or a combination thereof, thereby converting said free fatty acids to acetone.

The present disclosure also provides a method of producing acetone, comprising culturing a microorganism comprising reduced fadR expression or function, or increased expression of the fad regulon, increased expression of the atoDAEB genes, increased acetyl-CoA acetyltransferase expression or function, increased acetoacetyl-CoA transferase expression or function, and increased acetoacetate decarboxylase expression or function, in a culture medium comprising free fatty acids, monoglycerides, diglycerides, triglycerides, phospholipids, or a combination thereof, thereby producing acetone. Recovery of acetone can be accomplished by standard techniques known to those of skill in the art, including, but not limited to, recovery from the fermentation broth by vacuum distillation or liquid-liquid extraction.

The microorganism may comprise further genes for the conversion of the acetone into other chemicals. For example, the microorganism may further comprise a gene encoding an acetone monooxygenase for the conversion of the acetone to methyl acetate. Thus, the present invention provides a microorganism comprising reduced fadR expression or function, or increased expression of the fad regulon, increased expression of the atoDAEB genes, increased acetyl-CoA acetyltransferase expression or function, increased acetoacetyl-CoA transferase expression or function, increased acetoacetate decarboxylase expression or function, and a gene encoding an acetone monooxygenase. In certain embodiments, the acetone monooxygenase is AcmA from Gordonia sp. Strain TY-5.

The present disclosure therefore provides a method of converting fatty acids to methyl acetate, comprising culturing a microorganism comprising reduced fadR expression or function, or increased expression of the fad regulon, increased expression of the atoDAEB genes, increased acetyl-CoA acetyltransferase expression or function, increased acetoacetyl-CoA transferase expression or function, increased acetoacetate decarboxylase expression or function, and a gene encoding an acetone monooxygenase, in a culture medium comprising free fatty acids, monoglycerides, diglycerides, triglycerides, phospholipids, or a combination thereof, thereby converting said free fatty acids to methyl acetate.

The present disclosure also provides a method of producing methyl acetate, comprising culturing a microorganism comprising reduced fadR expression or function, or increased expression of the fad regulon, increased expression of the atoDAEB genes, increased acetyl-CoA acetyltransferase expression or function, increased acetoacetyl-CoA transferase expression or function, increased acetoacetate decarboxylase expression or function, and a gene encoding an acetone monooxygenase, in a culture medium comprising free fatty acids, monoglycerides, diglycerides, triglycerides, phospholipids, or a combination thereof, thereby producing methyl acetate. Recovery of methyl acetate can be accomplished by standard techniques known to those of skill in the art.

Alternatively, the microorganism further comprises a gene encoding a secondary alcohol dehydrogenase for the conversion of the acetone to isopropanol. The present disclosure therefore provides a microorganism comprising reduced fadR expression or function, or increased expression of the fad regulon, increased expression of the atoDAEB genes, increased acetyl-CoA acetyltransferase expression or function, increased acetoacetyl-CoA transferase expression or function, increased acetoacetate decarboxylase expression or function, and a gene encoding secondary alcohol dehydrogenase. In particular embodiments, the secondary alcohol dehydrogenase is Sadh from Clostridium beijerinckii or Adh from Thermoanaerobacter brockii.

The present disclosure therefore provides a method of converting fatty acids to isopropanol, comprising culturing a microorganism comprising reduced fadR expression or function, or increased expression of the fad regulon, increased expression of the atoDAEB genes, increased acetyl-CoA acetyltransferase expression or function, increased acetoacetyl-CoA transferase expression or function, increased acetoacetate decarboxylase expression or function, and a gene encoding a secondary alcohol dehydrogenase, in a culture medium comprising free fatty acids, monoglycerides, diglycerides, triglycerides, phospholipids, or a combination thereof, thereby converting said free fatty acids to isopropanol.

The present disclosure also provides a method of producing isopropanol, comprising culturing a microorganism comprising reduced fadR expression or function, or increased expression of the fad regulon, increased expression of the atoDAEB genes, increased acetyl-CoA acetyltransferase expression or function, increased acetoacetyl-CoA transferase expression or function, increased acetoacetate decarboxylase expression or function, and a gene encoding a secondary alcohol dehydrogenase, in a culture medium comprising free fatty acids, monoglycerides, diglycerides, triglycerides, phospholipids, or a combination thereof, thereby producing isopropanol. Recovery of isopropanol can be accomplished by standard techniques known to those of skill in the art, including, but not limited to, recovery from the fermentation broth by vacuum distillation or liquid-liquid extraction.

Alternatively, the microorganism further comprises a gene encoding an acetol monooxygenase and either a gene encoding a glycerol dehydrogenase or genes encoding an acetol kinase, an L-1,2-propanediol-1-phosphate dehydrogenase, and a glycerol-1-phosphate phosphatase for the conversion of the acetone to 1,2-propanediol. Thus, the present disclosure also provides a microorganism comprising reduced fadR expression or function, or increased expression of the fad regulon, increased expression of the atoDAEB genes, increased acetyl-CoA acetyltransferase expression or function; increased acetoacetyl-CoA transferase expression or function, increased acetoacetate decarboxylase expression or function, and a gene encoding an acetol monooxygenase and a gene encoding a glycerol dehydrogenase, or a gene encoding an acetol monooxygenase; a gene encoding an acetol kinase; a gene encoding an L-1,2-propanediol-1-phosphate dehydrogenase; and, a gene encoding a glycerol-1-phosphate phosphatase. In certain embodiments, the microorganism comprises a gene encoding an acetol monooxygenase and a gene encoding a glycerol dehydrogenase. While acetol monooxygenase and glycerol dehydrogenase from any source can be utilized, in particular embodiments the acetol monooxygenase is the ethanol-inducible P-450 Isozyme 3a from rabbit, and the glycerol dehydrogenase is GldA from E. coli. In additional embodiments, the microorganism comprises a gene encoding an acetol monooxygenase, a gene encoding an acetol kinase, a gene encoding an L-1,2-propanediol-1-phosphate dehydrogenase, and a gene encoding a glycerol-1-phosphate phosphatase.

The present disclosure therefore provides a method of converting fatty acids to 1,2-propanediol, comprising culturing a microorganism comprising, reduced fadR expression or function, or increased expression of the fad regulon, increased expression of the atoDAEB genes, increased acetyl-CoA acetyltransferase expression or function, increased acetoacetyl-CoA transferase expression or function, increased acetoacetate decarboxylase expression or function, and a gene encoding an acetol monooxygenase and a gene encoding a glycerol dehydrogenase, or a gene encoding an acetol monooxygenase; a gene encoding an acetol kinase; a gene encoding an L-1,2-propanediol-1-phosphate dehydrogenase; and, a gene encoding a glycerol-1-phosphate phosphatase, in a culture medium comprising free fatty acids, monoglycerides, diglycerides, triglycerides, phospholipids, or a combination thereof, thereby converting said free fatty acids to 1,2-propanediol.

The present disclosure further provides a method of producing 1,2-propanediol, comprising culturing a microorganism comprising reduced fadR expression or function, or increased expression of the fad regulon, increased expression of the atoDAEB genes, increased acetyl-CoA acetyltransferase expression or function, increased acetoacetyl-CoA transferase expression or function, increased acetoacetate decarboxylase expression or function, and a gene encoding an acetol monooxygenase and a gene encoding a glycerol dehydrogenase, or a gene encoding an acetol monooxygenase; a gene encoding an acetol kinase; a gene encoding an L-1,2-propanediol-1-phosphate dehydrogenase, and a gene encoding a glycerol-1-phosphate phosphatase, in a culture medium comprising free fatty acids, monoglycerides, diglycerides, triglycerides, phospholipids, or a combination thereof, thereby producing 1,2-propanediol. Recovery of 1,2-propanediol can be accomplished by standard techniques known to those of skill in the art, including, but not limited to, recovery from the fermentation broth by vacuum distillation or liquid-liquid extraction.

In a further embodiment, the microorganism further comprises a gene overexpressing an acetyl-CoA acetyltransferase, a gene overexpressing a 3-hydroxybutyryl-CoA dehydrogenase, a gene overexpressing a crotonase, and a gene encoding a butyryl-CoA dehydrogenase. The microorganism may further comprise a gene overexpressing an acetyl-CoA:acetoacetyl-CoA transferase or a butyrate-acetoacetate CoA transferase, or both, and the high value chemical produced by the microorganism is butyrate. The present disclosure therefore provides a microorganism comprising reduced fadR expression or function, or increased expression of the fad regulon, increased expression of the atoDAEB genes, increased acetyl-CoA acetyltransferase expression or function, increased 3-hydroxybutyryl-CoA dehydrogenase expression or function, increased crotonase expression or function, a gene encoding butyryl-CoA dehydrogenase, and increased acetyl-CoA:acetoacetyl-CoA transferase or butyrate-acetoacetate CoA transferase expression or function, or a gene encoding phosphotransbutyrylase and a gene encoding butyrate kinase. In certain embodiments, the microorganism comprises increased acetyl-CoA:acetoacetyl-CoA transferase and butyrate-acetoacetate CoA transferase expression or function. While any source of 3-hydroxybutyrrl-CoA dehydrogenase, crotonase, and butyryl-CoA dehydrogenase can be utilized, in particular embodiments the 3-hydroxybutyryl-CoA dehydrogenase is Hbd from Clostridium acetobutylicum, the crotonase is from Clostridium acetobutylicum, and the butyryl-CoA dehydrogenase is Bcd from Clostridium acetobutylicum.

The disclosure thus provides a method of converting fatty acids to butyrate, comprising culturing a microorganism comprising reduced fadR expression or function, or increased expression of the fad regulon, increased expression of the atoDAEB genes, increased acetyl-CoA acetyltransferase expression or function, increased 3-hydroxybutyryl-CoA dehydrogenase expression or function, increased crotonase expression or function, a gene encoding butyryl-CoA dehydrogenase, and increased acetyl-CoA:acetoacetyl-CoA transferase or butyrate-acetoacetate CoA transferase expression or function, or a gene encoding phosphotransbutyrylase and a gene encoding butyrate kinase, in a culture medium comprising free fatty acids, monoglycerides, diglycerides, triglycerides, phospholipids, or a combination thereof, thereby converting said free fatty acids to butyrate.

The present disclosure also provides a method of producing butyrate, comprising culturing a microorganism comprising reduced fadR expression or function, or increased expression of the fad regulon, increased expression of the atoDAEB genes, increased acetyl-CoA acetyltransferase expression or function, increased 3-hydroxybutyryl-CoA dehydrogenase expression or function, increased crotonase expression or function, a gene encoding butyryl-CoA dehydrogenase, and increased acetyl-CoA:acetoacetyl-CoA transferase or butyrate-acetoacetate CoA transferase expression or function, or a gene encoding phosphotransbutyrylase and a gene encoding butyrate kinase, in a culture medium comprising free fatty acids, monoglycerides, diglycerides, triglycerides, phospholipids, or a combination thereof, thereby producing butyrate. Recovery of butyrate can be accomplished by standard techniques known to those of skill in the art, including, but not limited to, recovery from the fermentation broth by ion exchange, crystallization, or precipitation.

Alternatively, the microorganism further comprises a gene overexpressing a butyraldehyde dehydrogenase and a gene overexpressing a butanol dehydrogenase or secondary alcohol dehydrogenase, and the high value chemical that is produced is butanol. The present disclosure thus provides a microorganism comprising reduced fadR expression or function, or increased expression of the fad regulon, increased expression of the atoDAEB genes, increased acetyl-CoA acetyltransferase expression or function, increased 3-hydroxybutyryl-CoA dehydrogenase expression or function, increased crotonase expression or function, a gene encoding butyryl-CoA dehydrogenase, a gene encoding butyraldehyde dehydrogenase, and a gene encoding butanol dehydrogenase or secondary alcohol dehydrogenase. In certain embodiments, the microorganism comprises a gene encoding butanol dehydrogenase, while in other embodiments, the microorganism comprises a gene encoding secondary alcohol dehydrogenase. In particular embodiments, the butyraldehyde dehydrogenase is Bydh from Clostridium acetobutylicum, and the butanol dehydrogenase is Bdh from Clostridium acetobutylicum.

In certain aspects, the microorganism further comprises reduced NADH dehydrogenase expression or activity, for example by deletion of all or a portion of the NADH dehydrogenase gene.

The present disclosure thus provides a method of converting fatty acids to butanol, comprising culturing a microorganism comprising reduced fadR expression or function, or increased expression of the fad regulon, increased expression of the atoDAEB genes, increased acetyl-CoA acetyltransferase expression or function, increased 3-hydroxybutyryl-CoA dehydrogenase expression or function, increased crotonase expression or function, a gene encoding butyryl-CoA dehydrogenase, a gene encoding butyraldehyde dehydrogenase, and a gene encoding butanol dehydrogenase or secondary alcohol dehydrogenase, in a culture medium comprising free fatty acids, monoglycerides, diglycerides, triglycerides, phospholipids, or a combination thereof, thereby converting said free fatty acids to butanol.

The present disclosure further provides a method of producing butanol, comprising culturing a microorganism comprising reduced fadR expression or function, or increased expression of the fad regulon, increased expression of the atoDAEB genes, increased acetyl-CoA acetyltransferase expression or function, increased 3-hydroxybutyryl-CoA dehydrogenase expression or function, increased crotonase expression or function, a gene encoding butyryl-CoA dehydrogenase, a gene encoding butyraldehyde dehydrogenase, and a gene encoding butanol dehydrogenase or secondary alcohol dehydrogenase, in a culture medium comprising free fatty acids, monoglycerides, diglycerides, triglycerides, phospholipids, or a combination thereof, thereby producing butanol. Recovery of butanol can be accomplished by standard techniques known to those of skill in the art, including, but not limited to, recovery from the fermentation broth by vacuum distillation, liquid-liquid extraction, or pervaporation.

In another embodiment, the microorganism further comprises a gene overexpressing an acetyl-CoA acetyltransferase, a gene overexpressing a 3-hydroxy-3-methylglutaryl-CoA synthase, and a gene overexpressing a 3-hydroxy-3-methylglutaryl-CoA reductase. The high value chemical produced by the microorganism is mevalonate. Therefore, the present disclosure additionally provides a microorganism comprising reduced fadR expression or function, or increased expression of the fad regulon, increased expression of the atoDAEB genes, increased acetyl-CoA acetyltransferase expression or function, a gene encoding 3-hydroxy-3-methylglutaryl-CoA synthase, and a gene encoding 3-hydroxy-3-methylglutaryl-CoA reductase. Although any source of 3-hydroxy-3-methylglutaryl-CoA synthase or 3-hydroxy-3-methylglutaryl-CoA reductase can be utilized, in particular embodiments the 3-hydroxy-3-methylglutaryl-CoA synthase is Erg13 from Saccharomyces cerevisiae or Mva1 from Arabidopsis thaliana, and the 3-hydroxy-3-methylglutaryl-CoA reductase is HMG1 or HMG2 from Saccharomyces cerevisiae or HMG-CoA reductase from Lactobacillus reuteri.

The present disclosure therefore provides a method of converting fatty acids to mevalonate, comprising culturing a microorganism comprising reduced fadR expression or function, or increased expression of the fad regulon, increased expression of the atoDAEB genes, increased acetyl-CoA acetyltransferase expression or function, a gene encoding 3-hydroxy-3-methylglutaryl-CoA synthase, and a gene encoding 3-hydroxy-3-methylglutaryl-CoA reductase, in a culture medium comprising free fatty acids, monoglycerides, diglycerides, triglycerides, phospholipids, or a combination thereof, thereby converting said free fatty acids to mevalonate.

The present disclosure additionally provides a method of producing mevalonate, comprising culturing a microorganism comprising, reduced fadR expression or function, or increased expression of the fad regulon, increased expression of the atoDAEB genes, increased acetyl-CoA acetyltransferase expression or function, a gene encoding 3-hydroxy-3-methylglutaryl-CoA synthase, and a gene encoding 3-hydroxy-3-methylglutaryl-CoA reductase, in a culture medium comprising free fatty acids, monoglycerides, diglycerides, triglycerides, phospholipids, or a combination thereof, thereby producing mevalonate. Recovery of mevalonate can be accomplished by standard techniques known to those of skill in the art.

In a further embodiment, the microorganism further comprises a gene overexpressing an acetaldehyde dehydrogenase, and genes encoding the regulatory and catalytic subunits of ethanolamine ammonia-lyase. The high value chemical produced by the microorganism is ethanolamine. The present disclosure thus provides a microorganism comprising reduced fadR expression or function, or increased expression of the fad regulon, increased expression of the atoDAEB genes, increased acetaldehyde dehydrogenase expression or function, and genes encoding the regulatory and catalytic subunits of ethanolamine ammonia-lyase. In certain embodiments, the microorganism comprises an endogenous or heterologous acetaldehyde dehydrogenase. In particular embodiments, the acetaldehyde dehydrogenase is the mhpF gene from E. coli. In other embodiments, the regulatory subunit of ethanolamine ammonia-lyase is the eutB gene and the catalytic subunit of ethanolamine ammonia-lyase is the eutC gene.

The present disclosure thus provides a method of converting fatty acids to ethanolamine, comprising culturing a microorganism comprising, reduced fadR expression or function, or increased expression of the fad regulon, increased expression of the atoDAEB genes, increased acetaldehyde dehydrogenase expression or function, and genes encoding the regulatory and catalytic subunits of ethanolamine ammonia-lyase, in a culture medium comprising free fatty acids, monoglycerides, diglycerides, triglycerides, phospholipids, or a combination thereof, thereby converting said free fatty acids to ethanolamine. In certain embodiments, the culture medium further comprises a source of ammonia.

The present disclosure further provides a method of producing ethanolamine, comprising culturing a microorganism comprising reduced fadR expression or function, or increased expression of the fad regulon, increased expression of the atoDAEB genes, increased acetaldehyde dehydrogenase expression or function, and genes encoding the regulatory and catalytic subunits of ethanolamine ammonia-lyase, in a culture medium comprising free fatty acids, monoglycerides, diglycerides, triglycerides, phospholipids, or a combination thereof, thereby producing ethanolamine. In certain embodiments, the culture medium further comprises a source of ammonia. Recovery of ethanolamine can be accomplished by standard techniques known to those of skill in the art.

Thus, the present disclosure provides a microorganism comprising reduced fadR expression or function, or increased expression of the fad regulon, increased expression of the atoDAEB genes, and one or more of the following: (1) a nucleic acid sequence encoding an alcohol dehydrogenase protein that is active under aerobic conditions; (2) increased expression of an acetate kinase gene; (3) increased expression of a phosphate acetyltransferase gene; (4) increased expression of an ADP-forming acetyl-CoA synthetase activity; (5) expression of an acetyl-CoA synthetase activity that functions primarily to produce acetate from acetyl-CoA; (6) reduced sucA expression or function; (7) reduced sucB expression or function; (8) reduced sdhA expression or function ; (9) reduced sdhB expression or function; (10) increased expression or function of succinic semialdehyde dehydrogenase; (11) increased expression or function of succinyl-CoA:CoA transferase; (12) increased expression or function of succinate-CoA ligase; (13) increased gamma-hydroxybutyric acid dehydrogenase expression or function; (14) increased lactonase expression or function; (15) increased aldehyde dehydrogenase expression or function; (16) increased alcohol dehydrogenase expression or function; (17) increased methylmalonyl-CoA mutase expression or function; (18) increased methylmalonyl-CoA decarboxylase expression or function; (19) increased propionyl-CoA:succinate CoA transferase expression or function; (20) increased acetyl-CoA acetyltransferase expression or function; (21) increased acetoacetyl-CoA transferase expression or function; (22) increased acetoacetate decarboxylase expression or function; (23) a gene encoding an acetone monooxygenase; (24) a gene encoding secondary alcohol dehydrogenase; (25) a gene encoding an acetol monooxygenase; (26) a gene encoding a glycerol dehydrogenase; (27) a gene encoding an acetol kinase; (28) a gene encoding an L-1,2-propanediol-1-phosphate dehydrogenase; (29) a gene encoding a glycerol-1-phosphate phosphatase; (30) increased 3-hydroxybutyryl-CoA dehydrogenase expression or function; (31) increased crotonase expression or function; (32) a gene encoding butyryl-CoA dehydrogenase; (33) increased acetyl-CoA:acetoacetyl-CoA transferase expression or function; (34) increased butyrate-acetoacetate CoA transferase expression or function; (35) a gene encoding phosphotransbutyrylase; (36) a gene encoding butyrate kinase; (37) a gene encoding butyraldehyde dehydrogenase; (38) a gene encoding butanol dehydrogenase; (39) a gene encoding 3-hydroxy-3-methylglutaryl-CoA synthase; (40) a gene encoding 3-hydroxy-3-methylglutaryl-CoA reductase; (41) increased acetaldehyde dehydrogenase expression or function; (42) a gene encoding the regulatory subunit of ethanolamine ammonia-lyase; (43) a gene encoding the catalytic subunit of ethanolamine ammonia-lyase; (44) reduced NADH dehydrogenase expression or activity; (45) a nucleic acid sequence encoding a fadE protein that uses NAD+ or NADP+ as a co-factor; (46) reduced iclR expression or function; (47) increased expression of an aceA gene; or (48) increased expression of an aceB gene.

### BRIEF DESCRIPTION OF THE DRAWINGS

So that the manner in which the above recited features of the present disclosure can be understood in detail, a more particular description of the disclosure, briefly summarized above, may be had by reference to embodiments, some of which are illustrated in the appended drawings. It is to be noted, however, that the appended drawings illustrate only typical embodiments of this invention and are therefore not to be considered limiting of its scope, for the invention may admit to other equally effective embodiments.

FIG. 1 shows the pathway for the conversion of acetyl-CoA into ethanol by the AdhE protein. AdhE is tightly regulated to work only under anaerobic conditions. A mutant AdhE protein, denoted AdhE*, has escaped this regulation and will work only under aerobic conditions. This enzyme carries out two steps at one time, with the net consumption of two NADH per acetyl-CoA converted to ethanol.

FIG. 2 shows the pathway for the beta-oxidation of fatty acids. Multiple steps are indicated by broken arrows, single steps are indicated by solid arrows. Two reducing equivalents (one FADH2, one NADH) are produced through the β-oxidation cycle. When acyl(C=4)-CoA, the molecule is split into two acetyl-CoA molecules without the production of reducing equivalents.

FIG. 3 shows the role of a FadE protein with modified co-factor specificity in the beta-oxidation of fatty acids. Multiple steps are indicated by broken arrows, single steps are indicated by solid arrows. FadE cofactor specificity changed to use NAD+/NADH or NADP+/NADPH.

FIG. 4A and FIG. 4B show the pathway steps performed by the Pta and AckA proteins (FIG. 4A) and AcdA/AcdB proteins (FIG. 4B).

FIG. 5 shows a pathway for the production of succinate from fatty acids. Multiple steps are indicated by broken arrows, single steps are indicated by solid arrows. Step 1 is catalyzed by the enzyme 2-oxoglutarate dehydrogenase, and Step 2 is catalyzed by the enzyme succinate dehydrogenase.

FIG. 6 shows a pathway for the production of gamma-butyrolactone from succinate. Step 1 is catalyzed by the enzyme succinic semialdehyde dehydrogenase (E.C. 1.2.1.16 or 1.2.1.24), Step 2 is catalyzed by the enzyme succinyl-CoA:CoA transferase, Step 3 is catalyzed by the enzyme succinate-CoA ligase (ADP-forming; E.C. 6.2.1.5), Step 4 is catalyzed by the enzyme succinate semialdehyde dehydrogenase, Step 5 is catalyzed by the enzyme γ-hydroxybutyric acid dehydrogenase (E.C. 1.1.1.2 or 1.1.1.61), and Step 6 is catalyzed by the enzyme lactonase (E.C. 3.1.1.25).

FIG. 7 shows a pathway for the production of 1,4-butanediol from succinate. Step 1 is catalyzed by the enzyme succinic semialdehyde dehydrogenase (E.C. 1.2.1.16 or 1.2.1.24), Step 2 is catalyzed by the enzyme succinyl-CoA:CoA transferase, Step 3 is catalyzed by the enzyme succinate-CoA ligase (ADP-forming; E.C. 6.2.1.5), Step 4 is catalyzed by the enzyme succinate semialdehyde dehydrogenase, Step 5 is catalyzed by the enzyme γ-hydroxybutyric acid dehydrogenase (E.C. 1.1.1.2 or 1.1.1.61), Step 6 is catalyzed by the enzyme aldehyde dehydrogenase (E.C. 1.2.1.3, or 1.2.1.4), and Step 7 is catalyzed by the enzyme alcohol dehydrogenase (E.C. 1.1.1.202).

FIG. 8 shows a pathway for the production of propionate from succinate. Step 1 is catalyzed by the enzyme methylmalonyl-CoA mutase, Step 2 is catalyzed by the enzyme methylmalonyl-CoA decarboxylase, and Step 3 is catalyzed by the enzyme propionyl-CoA:succinate-CoA transferase.

FIG. 9 shows a pathway for the production of acetone from fatty acids. Multiple steps are indicated by broken arrows, single steps are indicated by solid arrows. Step 1 is catalyzed by the enzyme acetyl-CoA acetyltransferase (E.C. 6.2.1.1), Step 2 is catalyzed by the enzyme acetyl-CoA:acetoacetyl-CoA transferase (E.C. 2.8.3.X or 2.8.3.8), and Step 3 is catalyzed by the enzyme acetoacetate decarboxylase (E.C. 4.1.1.4).

FIG. 10 shows a pathway for the production of isopropanol from acetone. Step 1 is catalyzed by the enzyme secondary alcohol dehydrogenase (E.C. 1.1.1.80).

FIG. 11 shows a pathway for the production of 1,2-propanediol from acetone. Step 1 is catalyzed by the enzyme acetol monooxygenase (E.C. 1.14.14.1), Step 2 is catalyzed by the enzyme glycerol dehydrogenase (E.C. 1.1.1.6), Step 3 is catalyzed by the enzyme acetol kinase (E.C. 2.7.1.29), Step 4 is catalyzed by the enzyme L-1,2-propanediol-1-phosphate-dehydrogenase, and Step 5 is catalyzed by the enzyme glycerol-1-phosphate phosphatase.

FIG. 12 shows a pathway, including alternate steps, for the production of butyrate from fatty acids. Multiple steps are indicated by broken arrows, single steps are indicated by solid arrows. Alternate pathway indicated by dashed oval. Step 1 is catalyzed by the enzyme acetyl-CoA acetyltransferase (E.C. 6.2.1.1), Step 2 is catalyzed by the enzyme 3-hydroxybutryl-CoA dehydrogenase (E.C. 1.1.1.35), Step 3 is catalyzed by the enzyme crotonase (E.C. 4.2.1.55), Step 4 is catalyzed by the enzyme butyryl-CoA dehydrogenase (E.C. 1.3.99.2), Step 5 is catalyzed by the enzyme acetyl-CoA:acetoacetyl-CoA transferase (E.C. 2.8.3.X or 2.8.3.8), Step 6 is catalyzed by the enzyme phosphotransbutyrylase, and Step 7 is catalyzed by the enzyme butyrate kinase.

FIG. 13 shows a pathway for the production of butanol from fatty acids. Multiple steps are indicated by broken arrows, single steps are indicated by solid arrows. Step 1 is catalyzed by the enzyme acetyl-CoA acetyltransferase (E.C. 6.2.1.1), Step 2 is catalyzed by the enzyme 3-hydroxybutryl-CoA dehydrogenase (E.C. 1.1.1.35), Step 3 is catalyzed by the enzyme crotonase (E.C. 4.2.1.55), Step 4 is catalyzed by the enzyme butyryl-CoA dehydrogenase (E.C. 1.3.99.2), Step 5 is catalyzed by the enzyme butyraldehyde dehydrogenase (E.C. 1.2.1.57), and Step 6 is catalyzed by the enzyme butanol dehydrogenase.

FIG. 14 shows a pathway for the production of mevalonate from fatty acids. Multiple steps are indicated by broken arrows, single steps are indicated by solid arrows. Step 1 is catalyzed by the enzyme acetyl-CoA acetyltransferase (E.C. 6.2.1.1), Step 2 is catalyzed by the enzyme 3-hydroxy-3-methylglutaryl-CoA synthase (E.C. 2.3.3.10), and Step 3 is catalyzed by the enzyme 3-hydroxy-3-methylglutaryl-CoA reductase (E.C. 1.1.1.88).

FIG. 15 shows a pathway for the production of ethanolamine from fatty acids. Multiple steps are indicated by broken arrows, single steps are indicated by solid arrows. Step 1 is catalyzed by the enzyme acetaldehyde dehydrogenase (E.C. 1.2.1.10), and Step 2 is catalyzed by the enzyme ethanolamine ammonia-lyase (E.C. 4.3.1.7).

### DETAILED DESCRIPTION

It is to be understood that both the foregoing general description and the following detailed description are exemplary and explanatory only, and are not restrictive of the invention, as claimed. In this application, the use of the singular includes the plural, the word “a” or “an” means “at least one”, and the use of “or” means “and/or”, unless specifically stated otherwise. Furthermore, the use of the term “including”, as well as other forms, such as “includes” and “included”, is not limiting. Also, terms such as “element” or “component” encompass both elements or components comprising one unit and elements or components that comprise more than one unit unless specifically stated otherwise.

The section headings used herein are for organizational purposes only and are not to be construed as limiting the subject matter described. All documents, or portions of documents, cited in this application, including, but not limited to, patents, patent applications, articles, books, and treatises, are hereby expressly incorporated herein by reference in their entirety for any purpose. In the event that one or more of the incorporated literature and similar materials defines a term in a manner that contradicts the definition of that term in this application, this application controls.

Embodiments of the present disclosure provide microorganisms that are capable of efficient production of high-value chemicals. Suitable microorganisms, for example, may be selected from the group consisting of bacteria, yeast, and algae, either as wild type strains, mutant strains derived by classic mutagenesis and selection methods or as recombinant strains. Microorganisms that may be used include, but are not limited to: Escherichia coli, Lactobacillus spp., Lactococcus spp., Bacillus spp., Paenibacillus spp., Klebsiella spp., Citrobacter spp., Clostridium spp., Saccharomyces spp., Pichia spp., Zymomonas mobilis, Schizosaccharomyces pombe, and other suitable microorganisms known in the art. The high value chemicals include but are not limited to ethanol, acetate, succinate, gamma-butyrolactone, 1,4-butanediol, acetone, isopropanol, butyrate, butanol, mevalonate, propionate, ethanolamine, or 1,2-propanediol.

According to embodiments of the disclosure, the microorganisms are cultured on free fatty acids, monoglycerides, diglycerides, triglycerides, phospholipids, or combinations thereof as the primary carbon source or feedstock. The free fatty acids, monoglycerides, diglycerides, triglycerides, phospholipids, or combinations thereof may be provided by various sources, such as vegetable oils or animal fats.

As defined herein, a knocked-out gene is a gene whose encoded product, e.g., a protein, does not or substantially does not perform its usual function or any function. A knocked-out gene can be created through deletion, disruption, insertion, or mutation. As defined herein, microorganisms that lack one or more indicated knocked-out genes are also considered to have knock outs of the indicated gene(s). The microorganisms themselves may also be referred to as knock outs of the indicated gene(s). Such knock outs can also be conditional or inducible, using techniques that are well-known to those of skill in the art. Also contemplated are “knock ins”, in which a gene, or one or more segments of a gene, are introduced into the microorganism in place of, or in addition to, the endogenous copy of the gene. Once again, many techniques for creating knock in microorganisms are known to those of ordinary skill in the art.

The methods and techniques utilized for generating the microorganisms disclosed herein are known to the skilled worker trained in microbiological and recombinant DNA techniques. Methods and techniques for growing microorganisms (e.g., bacterial cells), transporting isolated DNA molecules into the host cell and isolating, cloning and sequencing isolated nucleic acid molecules, knocking out expression of specific genes, etc., are examples of such techniques and methods. These methods are described in many items of the standard literature, which are incorporated herein in their entirety: “Basic Methods In Molecular Biology” (Davis, et al., eds. McGraw-Hill Professional, Columbus, Ohio, 1986); Miller, “Experiments in Molecular Genetics” (Cold Spring Harbor Laboratory Press, Cold Spring Harbor, N.Y., 1972); Miller, “A Short Course in Bacterial Genetics” (Cold Spring Harbor Laboratory Press, Cold Spring Harbor, N.Y., 1992); Singer and Berg, “Genes and Genomes” (University Science Books, Mill Valley, Calif., 1991); “Molecular Cloning: A Laboratory Manual,” 2^{nd} Ed. (Sambrook, et al., eds., Cold Spring Harbor Laboratory Press, Cold Spring Harbor, N.Y., 1989); “Handbook of Molecular and Cellular Methods in Biology and Medicine” (Kaufman, et al., eds., CRC Press, Boca Raton, Fla., 1995); “Methods in Plant Molecular Biology and Biotechnology” (Glick and Thompson, eds., CRC Press, Boca Raton, Fla., 1993); and Smith-Keary, “Molecular Genetics of Escherichia coli” (The Guilford Press, New York, N.Y., 1989).

The skilled person will know how to reduce or abolish the activity of a gene or protein as described herein. Such may be for instance accomplished by either genetically modifying the host organism in such a way that it produces less or no copies of the protein than the wild type organism, or by decreasing or abolishing the specific activity of the protein. Modifications in order to have the organism produce less or no copies of the gene and/or protein may include the use of a weak promoter, or the mutation (e.g., insertion, deletion or point mutation) of (parts of) the gene or its regulatory elements. Decreasing or abolishing the specific activity of a protein may also be accomplished by methods known in the art. Such methods may include the mutation (e.g., insertion, deletion or point mutation) of (parts of) the gene. Also known in the art are methods of reducing or abolishing the activity of a given protein by contacting the protein with specific inhibitors or other substances that specifically interact with the protein. Potential inhibiting compounds may for instance be monoclonal or polyclonal antibodies against the protein. Such antibodies may be obtained by routine immunization protocols of suitable laboratory animals.

The highly reduced nature of carbon atoms in fatty acids (FAs), as compared to sugars, provides significant advantages in the production of many chemicals/fuels via fermentation. Table 1 provides an analysis of redox balance for the conversion of fatty acids (FAs) into cell mass and ethanol, with calculations for dodecanoic (C₁₂), tetradecanoic (C₁₄), hexadecanoic (C₁₆), and octadecanoic (C₁₈) acids shown (all saturated FAs).

TABLE 1

| | | |
|---|---|---|
| Pathway^{a} | Stoichiometry^{b} (k^{c}) | Δκ^{d} (H^{e}) |

Synthesis of cell mass from FAs

| | | |
|---|---|---|
| | C₁₂₋₁₈H₂₄₋₃₆O₂(68/12-104/18) | 16.4-26.6 |
| C₁₂-C₁₈ saturated FA→cell mass | →12-18CH_{1.9}O_{0.5}N_{0.2}(4.3)^{f} | (8.2-13.3H) |

FAs to ethanol: carbon-constrained scenario (no energy/redox constraint: 100% carbon yield)^{g}

| | | |
|---|---|---|
| C₁₂-C₁₈ saturated FA→ethanol | C₁₂₋₁₈H₂₄₋₃₆O₂(68/12-104/18)→6-9C₂H₆O(6) | -4 (-2H) |

FAs to ethanol: carbon- and redox-constrained scenario (94-96% carbon yield)^{h}

| | | |
|---|---|---|
| C₁₂ saturated FA→ethanol + CO₂ | C₁₂H₂₄O₂(68/12)→17/3C₂H₆O(6) + ⅔CO₂(0) | 0 (0H) |
| C₁₄ saturated FA→ethanol + CO₂ | C₁₄H₂₈O₂(80/14)→20/3C₂H₆O(6) + ⅔CO₂(0) | 0 (0H) |
| C₁₆ saturated FA→ethanol + CO₂ | C₁₆H₃₂O₂(92/16)→23/3C₂H₆O(6) + ⅔CO₂(0) | 0 (0H) |
| C₁₈ saturated FA→ethanol + CO₂ | C₁₈H₃₆O₂ (104/18)→26/3C₂H₆O(6) + ⅔CO₂(0) | 0 (0H) |

FAs to ethanol: carbon-, redox-, and energy-constrained scenario (92-94% carbon yield)ⁱ

| | | |
|---|---|---|
| C₁₂ saturated FA→ethanol + CO₂ | C₁₂H₂₄O₂(68/12)→11/2C₂H₆O(6) + CO₂(0) | 2 (1H) |
| C₁₄ saturated FA→ethanol + CO₂ | C₁₄H₂₈O₂(80/14)→13/2C₂H₆O(6) + CO₂(0) | 2 (1H) |
| C₁₆ saturated FA→ethanol + CO₂ | C₁₆H₃₂O₂(92/16)→15/2C₂H₆O(6) + CO₂(0) | 2 (1H) |
| C₁₈ saturated FA→ethanol + CO₂ | C₁₈H₃₆O₂(104/18)→17/2C₂H₆O(6) + CO₂(0) | 2 (1H) |

^{a}The pathway mediating utilization of FAs is known as the FA β-oxidation pathway. Each two-carbon segment of a FA molecule generates a molecule of acetyl-CoA along with one equivalent each of FADH₂ and NADH. Both FADH₂ and NADH can be used in the synthesis of ATP via oxidative phosphorylation. Ethanol is synthesized in E. coli from acetyl-CoA in a two-step process catalyzed by the enzyme acetaldehyde/alcohol dehydrogenase, a pathway that consumes two reducing equivalents.
^{b}Pathway stoichiometry accounts only for carbon balance between reactants and products. ${\sum }_{over i reactants} {v}_{i} {c}_{i} {κ}_{i} - {\sum }_{over j products} {v}_{j} {c}_{i} {κ}_{j}$ ^{c}The degree of reduction per carbon, k, was estimated as described elsewhere (Nielsen, et al., “Bioreaction Engineering Principles,” 2^{nd} Ed., pp. 60-73, Springer, New York, NY, 2003).
^{d}Degree of reduction balance (Δκ) is estimated as ${\sum }_{over i reactants} {v}_{i} {c}_{i} {κ}_{i} - {\sum }_{over j products} {v}_{j} {c}_{i} {κ}_{j} ,$ where n and c are the stoichiometric coefficient and the number of carbon atoms for each compound, respectively.
^{e}Net redox units, H, are expressed per mole of fatty acid (H ≡ NAD(P)H = FADH₂ = “H₂”).
^{f}Cell mass formula is the average of reported for different microorganisms (Nielsen, et al., 2003, supra). Conversion of FAs into cell mass neglects carbon losses as 1-C metabolites. In consequence, the degree of reduction balance in this case represents the minimum amount of redox units generated.
^{g}This scenario assumes conversion of all AcCoA generated from FA oxidation into ethanol.
^{h}This scenario considers oxidation of ⅓ of an AcCoA molecule to CO₂ via TCA cycle, per each molecule of FA metabolized, which would provide the reducing equivalents required to operate the ethanol pathways.
ⁱThis scenario considers oxidation of ½ of an AcCoA molecule to CO₂ via TCA cycle, per each molecule of FA metabolized, which coupled to oxidative phosphorylation would provide the ATP and reducing equivalents required to operate the β-oxidation and ethanol pathways, respectively.

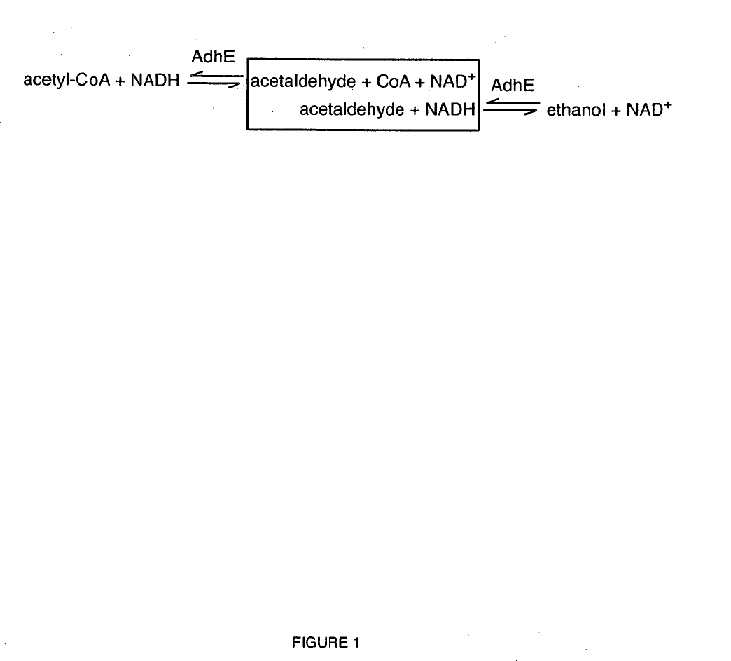

The degree of reduction per carbon of a 12 to 18 carbon FA ranges from 5.67 to 5.78 (Table 1), as compared to a degree of reduction of 4 for glucose (C₆H₁₂O₆: κ=4) or xylose (C₅H₁₀O₅: κ=4). The advantage of this higher degree of reduction is best illustrated by considering the production of a reduced compound, such as ethanol (Table 1). Ethanol is synthesized in E. coli from acetyl-CoA in a two-step process catalyzed by the acetaldehyde/alcohol dehydrogenase enzyme, a pathway that consumes two reducing equivalents (FIG. 1). The generation of one molecule of acetyl-CoA from a two-carbon fragment of a FA in each cycle through the β-oxidation pathway generates two reducing equivalents. Therefore, ethanol could be produced at a carbon yield approaching 100% because the two reducing equivalents generated in the synthesis of acetyl-CoA would be consumed in the production of ethanol (Table 1: carbon-constrained scenario); however, as the number of cycles through the β-oxidation pathway is always one less than the number of acetyl-CoA groups formed, each mole of FA generates (N/2)−1 moles of reducing equivalents (where N=the number of carbons in a saturated FA molecule). Energy and degree of reduction balances for the conversion of 12- to 18-carbon saturated FAs into ethanol yield a maximum theoretical yield of ethanol in the range of 92% to 94% on a carbon basis (Table 1: carbon-, redox, and energy-constrained scenario). The use of glucose or xylose as a fermentation substrate for the production of ethanol results in a shortage of reducing equivalents. This shortage limits the ethanol yield to a theoretical maximum of 67% on a carbon basis; e.g. for glucose C₆H₁₂O₆ (4)→2C₂H₆O (6)+2CO₂ (0). Comparison of ethanol yields from sugars and fatty acids is more relevant on a weight basis, as this is the metric used in the feedstocks and fuels/chemicals markets. On weight basis, the maximum theoretical yield of ethanol from FAs is approximately two-fold higher than the yield of ethanol from sugars (Table 2). Furthermore, in contrast to the production of ethanol from glucose, wherein approximately one-half of the glucose (by weight) is converted to carbon dioxide, the conversion of FAs to ethanol does not yield carbon dioxide as a significant co-product.

TABLE 2

| | Maximum theoretical | Maximum theoretical |
|---|---|---|
| | yield for | yield for |
| | EtOH (weight basis) | EtOH (carbon basis) |
| Sugars | 0.51 | 0.67 |
| Fatty Acids | 1.32 ± 0.05 | 0.93 |
| (C12-C18 saturated FA) | (2.6-fold higher) | (1.4-fold higher) |

These calculations are based on the assumption that a fraction of each FA molecule is metabolized through the citric acid cycle to provide for the energy and reducing equivalents required to produce ethanol from FAs (Table 1: carbon-, redox-, and energy-constrained scenarios). Therefore, carbon, redox, and energy constraints are accounted for.

Embodiments of the disclosure provide microorganisms capable of converting fatty acids into high-value chemicals and methods for the aerobic β-oxidation of fatty acids that result in the production of high-value chemicals, such as ethanol or succinate. Typically, the β-oxidation of fatty acids by prior microorganisms under aerobic conditions generates energy and carbon that is converted into more cell mass rather than high-value chemicals, and the production of high-value chemicals by prior microorganisms typically is attempted by fermentation of other carbon sources under anaerobic conditions. The genetic modifications set forth in the present disclosure allow the acetyl-CoA produced from fatty acids to be converted into high value chemicals, such as ethanol or succinate, under aerobic conditions. As defined herein, aerobic conditions include aerobic/respiratory conditions as well as microaerobic/microrespiratory conditions. In other words, the aerobic conditions may include various levels of oxygen or other electron acceptors, such as nitrate (NO₃) or nitrite (NO₂).

Microorganisms for converting fatty acids to ethanol. Certain microorganisms of the present disclosure comprise (1) a knocked-out fadR gene (Ecocyc Gene ID EG10281); (2) a mutation in an atoC (Ecocyc Gene ID EG 11668) gene that provides overexpression of the microorganism's ato operon; and (3) a gene expressing alcohol dehydrogenase activity under aerobic or microaerobic conditions.

Generally, fadR encodes a repressor that coordinately regulates fatty acid degradation, fatty acid biosynthesis, and acetate metabolism. More specifically, fadR encodes a dual DNA-binding transcriptional regulator protein for fatty acid metabolism. The protein belongs to the GntR family of transcriptional regulators. The protein controls (e.g., represses) the expression of several genes involved in fatty acid transport and β-oxidation, including fadBA (Ecocyc Gene ID EG10279 and EG10278, respectively), fadD (Ecocyc Gene ID EG11530),fadL (Ecocyc Gene ID EG10280), and fadE (Ecocyc Gene ID G6105). The protein also activates the transcription of at least three genes required for unsaturated fatty acid biosynthesis: fabA, fabB, and iclR (Ecocyc Gene ID EG10273, EG10274, and EG10491, respectively).

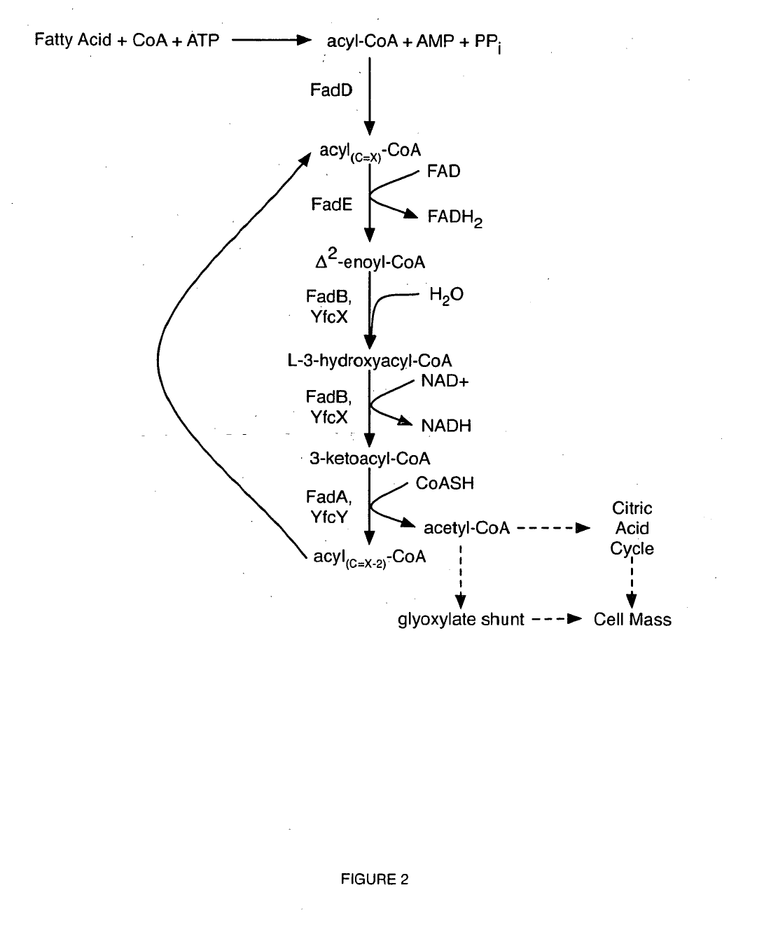

Microorganisms having a functional fadR gene, such as wild-type E. coli, will preferentially activate and digest long-chain fatty acids first. Once all of the long-chain fatty acids have been digested, E. coli will proceed to processing the medium-chain fatty acids. Loss of the fadR gene function allows the E. coli to degrade both long chain fatty acids, i.e., fatty acids having 12 or more carbons in the fatty acid chain, as well as medium chain fatty acids, i.e., fatty acids having 6-11 carbons in the fatty acid chain, at the same time, eliminating the problem of partial degradation of the available fatty acid pool and increasing the conversion yield of fatty acids to acetyl-CoA. As a result, a knock-out of fadR increases the pool of fatty acids for the fatty acid β-oxidation pathway depicted in FIG. 2.

Once the chain length of a fatty acid in the β-oxidation pathway falls to about six carbons or less, the β-oxidation pathway is no longer sufficient to efficiently degrade the fatty acid. Instead, the degradation is carried out by a separate enzymatic system encoded by the ato operon. Overexpression of the ato operon produces ato gene products that are required for the efficient conversion of the last 4 carbon fragments (4 carbon acyl-CoA) in the fatty acid chain into two molecules of acetyl-CoA without the generation of reducing equivalents. Certain embodiments of the disclosure comprise a mutation in the atoC gene that results in high constitutive expression of the ato operon to ensure efficient degradation of short chain fatty acids. The atoC gene encodes a transcriptional activator of the ato operon. An example of a mutation in atoC that results in high constitutive expression of the ato operon is described in Pauli and Overath (Eur. J. Biochem. 29:553-562, 1972), which is herein incorporated by reference.

Optionally, the microorganism may be further modified by reduction or elimination of the alcohol dehydrogenase gene. The alcohol dehydrogenase gene of Escherichia coli, adhE (Ecocyc Gene ID EG10031), for example, is tightly regulated to function only under anaerobic conditions. The regulatory system represses alcohol dehydrogenase activity at the transcriptional, translational, and post-translational levels. An example of a mutation in adhE that results in the aerobic conversion of acetyl-CoA to ethanol is described in Holland-Staley, et al. (J. Bact. 182:6049-6054, 2000), which is herein incorporated by reference. The mutant adhE gene may contain mutations in both the regulatory and coding regions. Alternatively, expression of the adhE gene or mutant adhE gene may be provided by the use of a constitutively expressed promoter, an inducible promoter, or an aerobically regulated promoter operationally linked to the adhE gene or mutant adhE gene.

Optionally, the microorganism may be further modified by reduction or elimination of the NADH dehydrogenase activity. Such a mutation would ensure that all NADH generated via β-oxidation is available for use in the synthesis of ethanol. FIG. 1 provides the biochemical steps for converting acetyl-CoA to ethanol.

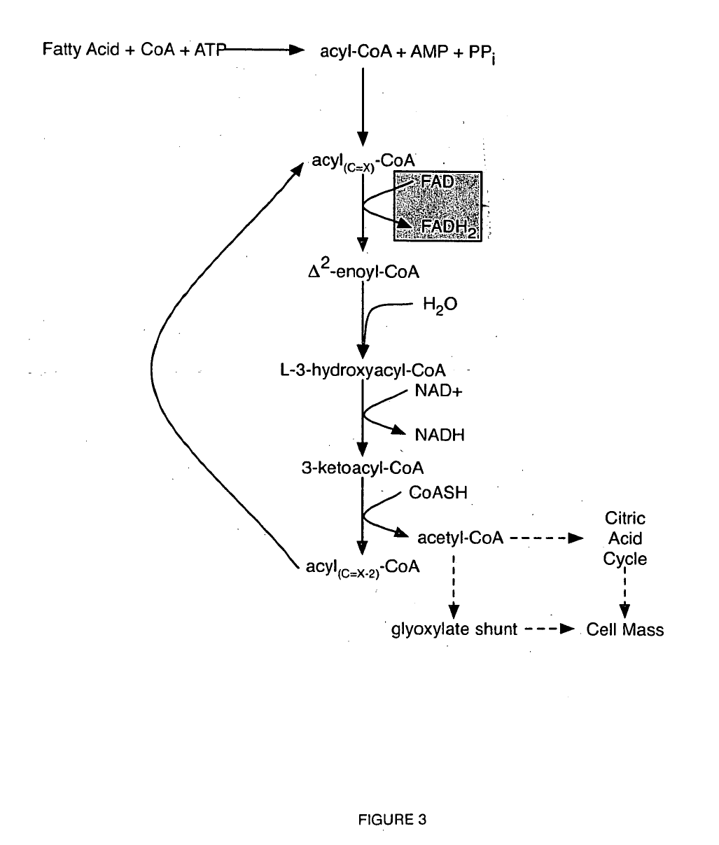

Optionally, the microorganism may further comprise a fadE gene encoding a protein that is capable of using NAD+ or NADP+ as its co-factor. As shown in FIG. 3, the fadE enzyme (E.C. 1.3.99.3) is involved in the conversion of acyl _{(C═X)}-CoA to Δ²-enoyl-CoA step in the cyclical β-oxidation of a fatty acid. The fadE gene may be an endogenous gene that has been mutated to alter its co-factor specificity from FAD to NAD+ or NADP+. Alternatively, the fadE gene may be a heterologous, overexpressed gene from an organism where the fadE gene product naturally uses NAD+ or NADP+, such as Mycobacterium smegmatis (Entrez GeneID: 4535406, GenBank Accession Number YP_{—}890239) or Euglena gracilis. In a further embodiment, a means distinct from fadE is used to transfer reducing power from the FADH₂ pool to the NAD(P)H pool. The generation of NADH by a fadE gene according to embodiments of the disclosure, rather than the generation of FADH₂ is desirable as the NADH reducing equivalents could be used in further processing of the acetyl-CoA produced by β-oxidation. The use of an altered FadE protein is particularly advantageous for the synthesis of molecules such as ethanol or butanol, where the NAD(P)H pool may be limiting in the absence of a FadE protein with altered co-factor specificity. For example, as shown in FIG. 1, 2 NADH molecules required for the conversion of acetyl-CoA into ethanol by AdhE could be provided by β-oxidation if the FadE co-factor specificity is altered to function with NAD(P)⁺ rather than FAD⁺. In general, NADH and NADPH are more useful than FADH₂ in the downstream pathways used to provide high-value chemicals of interest. Also, under certain conditions, such as in the absence of electron acceptors, FADH₂ generated during the β-oxidation cycle may accumulate to harmful levels.

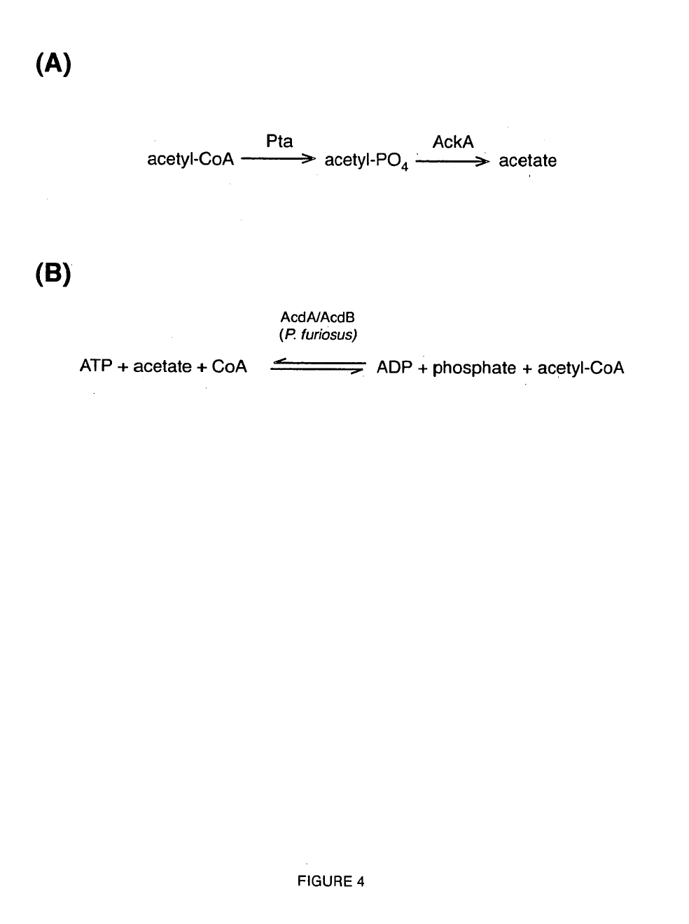

Microorganisms for converting fatty acids to acetate. In an alternate embodiment, microorganisms according to the present disclosure comprise (1) a knocked-out fadR gene; (2) a mutation in an atoC gene that provides overexpression of the microorganism's ato operon; and (3) a gene overexpressing ackA pta operon (Ecocyc Gene ID EG10027 and EG20173, respectively) or comparable genes encoding acetate kinase (E.C. 2.7.2.1) and phosphate acetyltransferase (E.C. 2.3.1.8) activities in concert to enhance the production of acetate. Optionally, the microorganism may comprise one of either a pta gene that overexpresses phosphate acetyltransferase or an ackA gene that overexpresses acetate kinase, that individually, serves to optimize acetate production from fatty acids. FIG. 4(A) provides a pathway for converting acetyl-CoA, derived from fatty acids, into acetate via AckA/Pta.

Alternatively, the microorganism comprises (1) a knocked-out fadR gene; (2) a mutation in an atoC gene that provides overexpression of the microorganism's ato operon; and (3) heterologous genes overexpressing the acdA and acdB genes of Pyrococcus furiosus (GenBank Accession Nos. AE010255 and AE010276). Together, these genes encode an ADP-forming acetyl-CoA synthetase activity (E.C. 6.2.1.13) that catalyzes the conversion of acetyl-CoA and phosphate to acetate and CoA-SH. Such a microorganism may, optionally, include a knocked-out gene encoding an endogenous acetyl-CoA synthetase activity that functions primarily to catalyze the reverse reaction. An example of an endogenous gene that preferentially catalyzes the reverse reaction is the acs gene (Ecocyc Gene ID EG11448) of Escherichia coli. Alternatively, the microorganism may comprise a mutant acetyl-CoA synthetase activity that favors the flow of acetyl-CoA to acetate. An exemplary use of such a microorganism is for the production of high levels of acetate from fatty acids. FIG. 4(B) provides an alternate pathway for converting acetyl-CoA, derived from fatty acids, into acetate.

Microorganisms for converting fatty acids to succinate. In an alternate embodiment, microorganisms according to the present disclosure comprise (1) a knocked-out fadR gene; (2) a mutation in an atoC gene that provides overexpression of the microorganism's ato operon; (3) a knocked-out sucA gene or sucB gene, or both (Ecocyc Gene ID EG10979 and EG10980, respectively); and (4) a knocked-out sdhA gene or sdhB gene, or both (Ecocyc Gene ID EG10931 and EG 10932). An exemplary use of such an organism is for the production of high levels of succinate from fatty acids.

The sdhAB operon encodes a succinate dehydrogenase that catalyzes the conversion of succinate to fumarate. Thus, knocking out the sdhAB operon favors the accumulation of succinate.

sucA and sucB encode required subunits of the 2-oxoglutarate dehydrogenase complex and catalyze the reaction alpha-ketoglutarate+coenzyme A+NAD⁺⇄succinyl-CoA+CO₂+NADH (E.C. 2.3.1.61). Knocking out the sucA and sucB genes opens the tricarboxylic acid cycle. The combination of opening the tricarboxylic acid cycle and activating/overexpressing the glyoxylate shunt results in the generation of one molecule of succinate for every two molecules of acetyl-CoA entering the glyoxylate shunt.

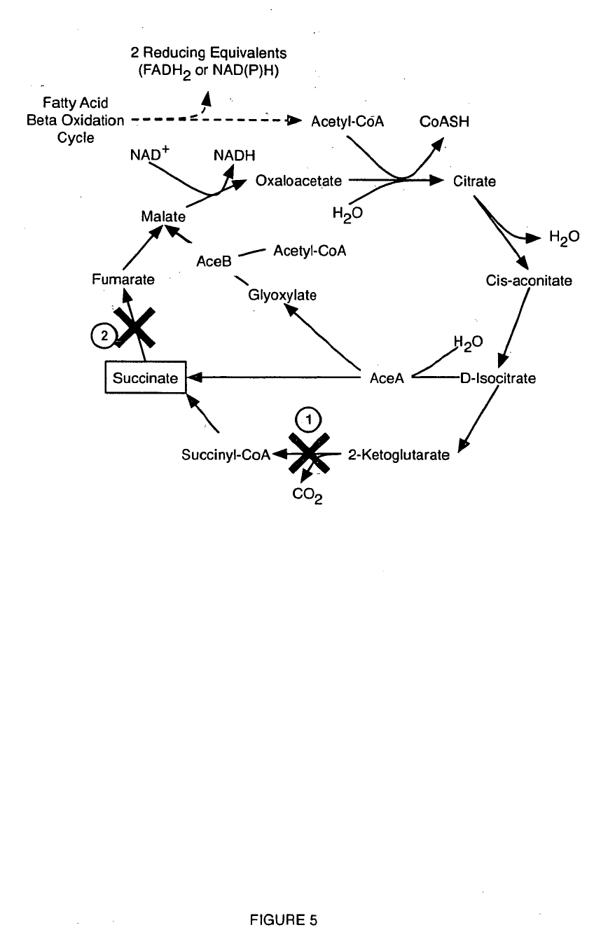

Optionally, the microorganism may also comprise a knock-out of the iclR gene (Ecocyc Gene ID EG10491). Knocking out the iclR gene activates the glyoxylate shunt, as the iclR gene product is a repressor of the aceBAK operon. The aceBAK operon includes the aceB, aceA, and aceK genes (Ecocy Gene ID EG10023, EG10022, and EG10026, respectively). aceB and aceA are involved in the glyoxylate shunt, as the aceA gene product converts isocitrate into glyoxylate and succinate, and the aceB gene product combines the glyoxylate with an acetyl-CoA to form malate, as shown in FIG. 5. The malate then continues through the remaining steps of the citric acid cycle (which is also known as the tricarboxylic acid cycle), where it can be processed with an additional acetyl-CoA into succinate and glyoxylate.

Microorganisms for converting fatty acids to gamma-butyrolactone. In an alternate embodiment, microorganisms according to the present disclosure comprise (1) a knocked-out fadR gene; (2) a mutation in an atoC gene that provides overexpression of the microorganism's ato operon; (3) a knocked-out sucA gene or sucB gene, or both; (4) a knocked-out sdhA gene or sdhB gene, or both; (5) a gene overexpressing a succinic semialdehyde dehydrogenase; (6) a gene overexpressing a gamma-hydroxybutyric acid dehydrogenase; and (7) a gene overexpressing a lactonase.

As an alternative to a succinic semialdehyde dehydrogenase that converts succinate to succinic semialdehyde, succinate may first be converted to succinyl-CoA by expression of genes encoding succinyl-CoA:CoA transferase (no E.C. number assigned), or by succinate-CoA ligase (E.C. 6.2.1.5). Overexpression or heterologous expression of a succinate semialdehyde dehydrogenase capable of converting succinyl-CoA to succinic semialdehyde completes the transformation of succinate to succinic semialdehyde.

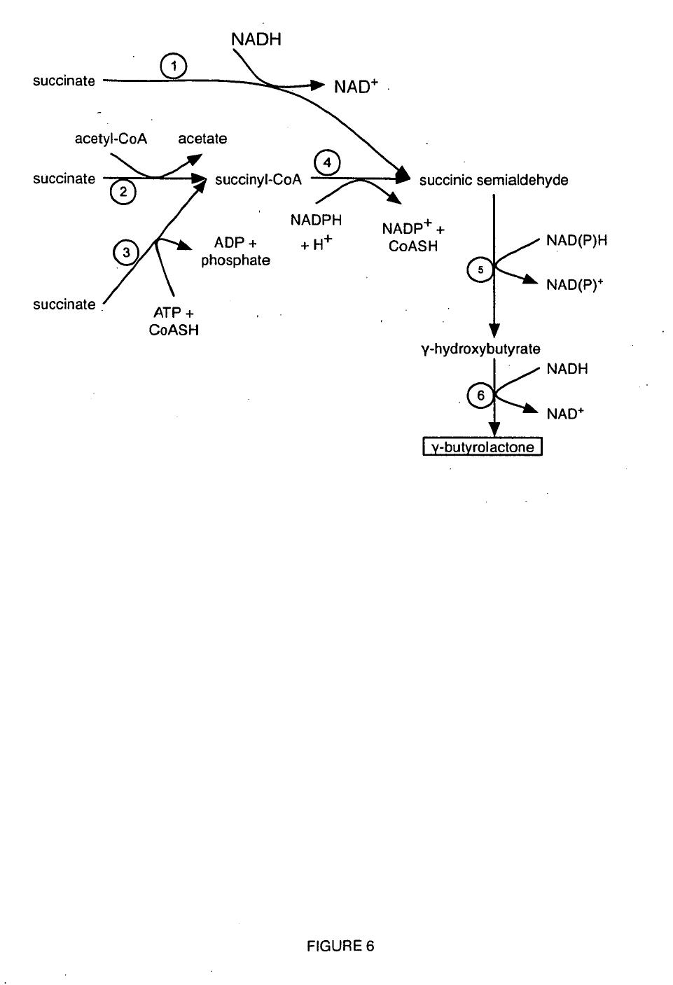

Optionally, the microorganism may be further modified by reduction or elimination of the NADH dehydrogenase activity. Such a mutation would ensure that all NADH generated via β-oxidation is available for use in the synthesis of gamma-butyrolactone. FIG. 6 shows a pathway for the conversion of succinate into gamma-butyrolactone and Table 3 provides exemplary enzymes for the steps in the pathway.

TABLE 3

| | |
|---|---|
| E.C. No. | Exemplary Enzyme(s) |

Succinic semialdehyde dehydrogenase

| | |
|---|---|
| 1.2.1.24 or 1.2.1.16 | AldA, E. coli (NAD⁺-linked) (Entrez GeneID: 945672, GenBank NP_415933) |
| | Sad (GabD, YneI), E. coli (NAD⁺-dependent) (Entrez GeneID: 947440, |
| | GenBank NP_416042) |
| | Gabd1, Mycobacterium tuberculosis (Entrez GeneID: 923143, GenBank |
| | NP_334651) |
| | AldH5A1, Homo sapiens (Entrez GeneID: 7915, GenBank NP_733936 or |
| | NP_001071) |
| | Ssadh1, Arabidopsis thaliana (Entrez GeneID: 844282, GenBank NP_178062) |
| | AttK, Agrobacterium tumefaciens (Entrez GeneID: 1136121, GenBank |
| | NP_356407) |

Succinyl-CoA:CoA transferase

| | |
|---|---|
| Unassigned | Succinyl-CoA:CoA transferase, Clostridium kluyveri (Entrez GeneID: 5392695, |
| | GenBank YP_001396395) |
| | Acetyl:Succinate CoA-transferase, Tritrichomonas foetus |
| | Acetyl:Succinate CoA-transferase, Trypanosoma brucei |
| 6.2.1.1 | Acetyl-CoA synthetase (AMP-forming), E. coli (Entrez GeneID: 948572, |
| | GenBank NP_418493) |

Succinate:CoA ligase (ADP-forming)

| | |
|---|---|
| 6.2.1.5 | Succinyl-CoA synthetase, E. coli (Entrez GeneID: 945312 and 945314, GenBank |
| | NP_415256 and NP_415257) |

Gamma-hydroxybutyric acid dehydrogenase

| | |
|---|---|
| 1.1.1.61 or 1.1.1.2 | 4-hydroxybutyrate dehydrogenase, NADPH-dependent, A. thaliana |
| | NADPH-dependent alcohol dehydrogenase from Bos taurus (Entrez |
| | GeneID: 281360, GenBank NP_777247), H. sapiens (Entrez GeneID: 10901, |
| | GenBank NP_066284), Rattus norvegicus, Oryctolagus cuniculus, or Sus scrofa |
| | BlcB, A. tumefaciens (Entrez GeneID: 1136911, GenBank NP_396070) |

Lactonase

| | |
|---|---|
| 3.1.1.25 | AttM, A. tumefaciens (also found in H. sapiens, Mus musculus) |
| | Lipase B48, Candida Antarctica |

Microorganisms for converting fatty acids to 1,4-butanediol. In an alternate embodiment, microorganisms according to the present disclosure comprise (1) a knocked-out fadR gene; (2) a mutation in an atoC gene that provides overexpression of the microorganism's ato operon; (3) a knocked-out sucA gene or sucB gene, or both; (4) a knocked-out sdhA gene or sdhB gene, or both; (5) a gene overexpressing a succinic semialdehyde dehydrogenase; (6) a gene overexpressing a gamma-hydroxybutyric acid dehydrogenase; (7) a gene overexpressing an aldehyde dehydrogenase, and (8) a gene overexpressing an alcohol dehydrogenase.

As an alternative to a succinic semialdehyde dehydrogenase that converts succinate to succinic semialdehyde, succinate may first be converted to succinyl-CoA by expression of genes encoding succinyl-CoA:CoA transferase (no E.C. number assigned), or by succinate-CoA ligase (E.C. 6.2.1.5). Overexpression or heterologous expression of a succinate semialdehyde dehydrogenase capable of converting succinyl-CoA to succinic semialdehyde completes the transformation of succinate to succinic semialdehyde.

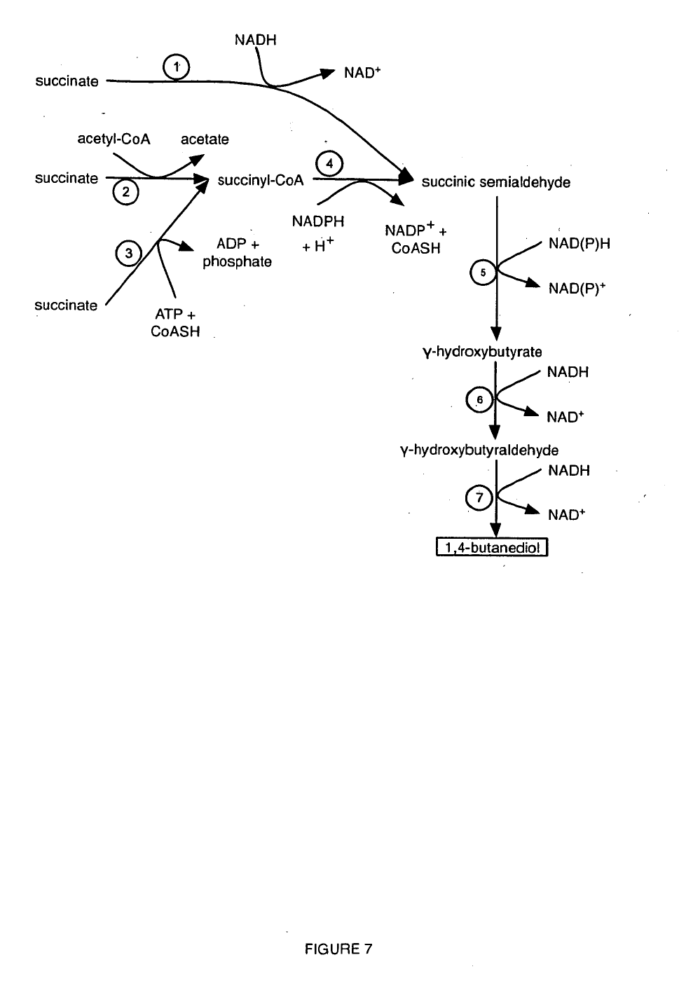

Optionally, the microorganism may be further modified by reduction or elimination of the NADH dehydrogenase activity. Such a mutation would ensure that all NADH generated via β-oxidation is available for use in the synthesis of 1,4-butanediol. FIG. 7 shows a pathway for the conversion of succinate into 1,4-butanediol and Tables 3 and 4 provide exemplary enzymes for the steps in the pathway.

TABLE 4

| | |
|---|---|
| E.C. No. | Exemplary Enzyme(s) |

Aldehyde dehydrogenase

| | |
|---|---|
| Unassigned | AldH (NAD(P)H-dependent dehydrogenase/gamma- |
| | glutamyl-gamma-aminobutyraldehyde dehydrogenase), |
| | E. coli (Entrez GeneID: 947003, GenBank NP_415816) |
| | Gamma-aminobutyraldehyde dehydrogenase (YdcW), |
| | E. coli (Entrez GeneID: 945876, GenBank NP_415961) |
| | TMP-1 aminobutyraldehyde dehydrogenase, |
| | Arthrobacter sp. |
| | Aminobutyraldehyde dehydrogenase (KauB), Pseudomonas |
| | aeruginosa |

Alcohol dehydrogenase

| | |
|---|---|
| 1.1.1.202 | 1,3-propanediol dehydrogenase, Citrobacter freundi or |
| | Klebsiella pneumoniae (Entrez GeneID: 6937135, |
| | GenBank YP_002236499) |

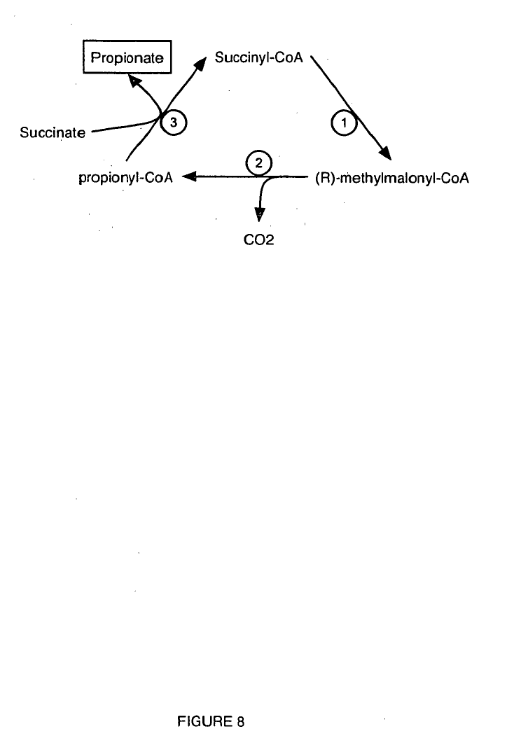

Microorganisms for converting fatty acids to propionate. In an alternate embodiment, microorganisms according to the present disclosure comprise (1) a knocked-out fadR gene; (2) a mutation in an atoC gene that provides overexpression of the microorganism's ato operon; (3) a knocked-out sucA gene or sucB gene, or both; (4) a knocked-out sdhA gene or sdhB gene, or both; (5) overexpression of a gene encoding a methylmalonyl-CoA mutase (scpA, Ecocyc Gene ID EG11444; Entrez GeneID: 945576); (6) overexpression of a gene encoding a methylmalonyl-CoA decarboxylase (scpB, Ecocyc Gene ID G7516; Entrez GeneID: 947408); and (7) overexpression of a gene encoding a propionyl-CoA:succinate CoA transferase activity (scpC, Ecocyc Gene ID G7517; Entrez GeneID: 947402). The overexpressed genes may either be endogenous or exogenous, or a combination thereof. FIG. 8 shows a pathway for the conversion of succinate into propionate.

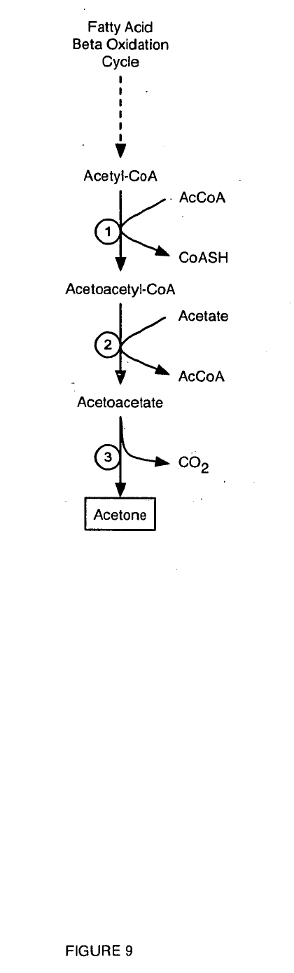

Microorganisms for converting fatty acids to acetone. In an alternate embodiment, microorganisms according to the present disclosure comprise (1) a knocked-out fadR gene; (2) a mutation in an atoC gene that provides overexpression of the microorganism's ato operon; (3) overexpression of a heterologous gene encoding an acetyl-CoA acetyltransferase or overexpression of an endogenous gene encoding an acetyl-CoA acetyltransferase; (4) a heterologous gene encoding an acetoacetyl-CoA transferase or overexpression of an endogenous gene encoding an acetoacetyl-CoA transferase; and (5) a heterologous gene encoding an acetoacetate decarboxylase. FIG. 9 shows a pathway for the conversion of acetyl-CoA into acetone and Table 5 shows exemplary enzymes for the steps in the pathway.

TABLE 5

| | |
|---|---|
| E.C. No. | Exemplary Enzyme(s) |

Acetyl-CoA acetyltransferase

| | |
|---|---|
| 6.2.1.1 | AtoB, E. coli (Entrez GeneID: 946727, |
| | GenBank NP_416728) |
| | Thiolase, Clostridium acetobutylicum |
| | (Entrez GeneID: 1119056, GenBank NP_349476) |

Acetyl-CoA:acetoacetyl-CoA transferase

| | |
|---|---|
| 2.8.3.—, 2.8.3.8 | CtfAB, C. acetobutylicum (Entrez GeneID: 1116168 |
| | and 1116169, GenBank NP_149326 and NP_149327) |
| | AtoAD, E. coli (Entrez GeneID: 946719 and 947525, |
| | GenBank NP_416726 and NP_416725) |

Acetoacetate decarboxylase

| | |
|---|---|
| 4.1.1.4 | Adc, C. acetobutylicum (Entrez GeneID: 1116170, |
| | GenBank NP_149328) |

Microorganisms for converting fatty acids to methyl acetate. In an alternate embodiment, microorganisms according to the present disclosure comprise (1) a knocked-out fadR gene; (2) a mutation in an atoC gene that provides overexpression of the microorganism's ato operon; (3) overexpression of a heterologous gene encoding an acetyl-CoA acetyltransferase or overexpression of an endogenous gene encoding an acetyl-CoA acetyltransferase; (4) a heterologous gene encoding an acetoacetyl-CoA transferase or overexpression of an endogenous gene encoding an acetoacetyl-CoA transferase; (5) a heterologous gene encoding an acetoacetate decarboxylase; and (6) a heterologous gene encoding an acetone monooxygenase, AcmA, from Gordonia sp. strain TY-5 (GenBank Acecssion Number AB252677 or BAF43791).

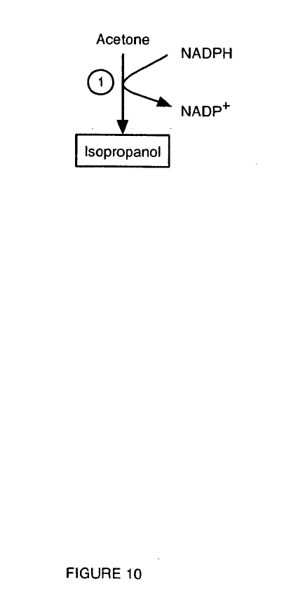

Microorganisms for converting fatty acids to isopropanol. In an alternate embodiment, microorganisms according to the present disclosure comprise (1) a knocked-out fadR gene; (2) a mutation in an atoC gene that provides overexpression of the microorganism's ato operon; (3) overexpression of a heterologous gene encoding an acetyl-CoA acetyltransferase or overexpression of an endogenous gene encoding an acetyl-CoA acetyltransferase; (4) a heterologous gene encoding an acetoacetyl-CoA transferase or overexpression of an endogenous gene encoding an acetoacetyl-CoA transferase; (5) a heterologous gene encoding an acetoacetate decarboxylase; and (6) a heterologous gene encoding a secondary alcohol dehydrogenase (E.C. 1.1.1.80). Examples of such alcohol dehydrogenases include include Sadh from Clostridium beijerinckii and Adh from Thermoanaerobacter brockii. FIG. 10 shows a pathway for the conversion of acetyl-CoA into isopropanol.

Microorganisms for converting fatty acids to 1,2-propanediol. In an alternate embodiment, microorganisms according to the present disclosure comprise (1) a knocked-out fadR gene; (2) a mutation in an atoC gene that provides overexpression of the microorganism's ato operon; (3) overexpression of a heterologous gene encoding an acetyl-CoA acetyltransferase or overexpression of an endogenous gene encoding an acetyl-CoA acetyltransferase; (4) a heterologous gene encoding an acetoacetyl-CoA transferase or overexpression of an endogenous gene encoding an acetoacetyl-CoA transferase; (5) a heterologous gene encoding an acetoacetate decarboxylase; (6) a heterologous gene encoding an acetol monooxygenase; and (6) overexpression of a homologous gene or heterologous gene encoding a glycerol dehydrogenase (E.C. 1.1.1.6). An example of an acetol monooxygenase is the ethanol-inducible P-450 Isozyme 3a of rabbit as described in Koop and Casazza (J. Biol. Chem. 260:13607-13612, 1985), which is herein incorporated by reference. The acetol monooxygenase (E.C. 1.14.14.1) catalyzes the conversion of acetone to acetol (dihydroxyacetone). An example of a glycerol dehydrogenase is the GldA enzyme of E. coli (Ecocyc Gene ID EG11904; Entrez GeneID: 948440; GenBank Accession Number NP_{—}418380).

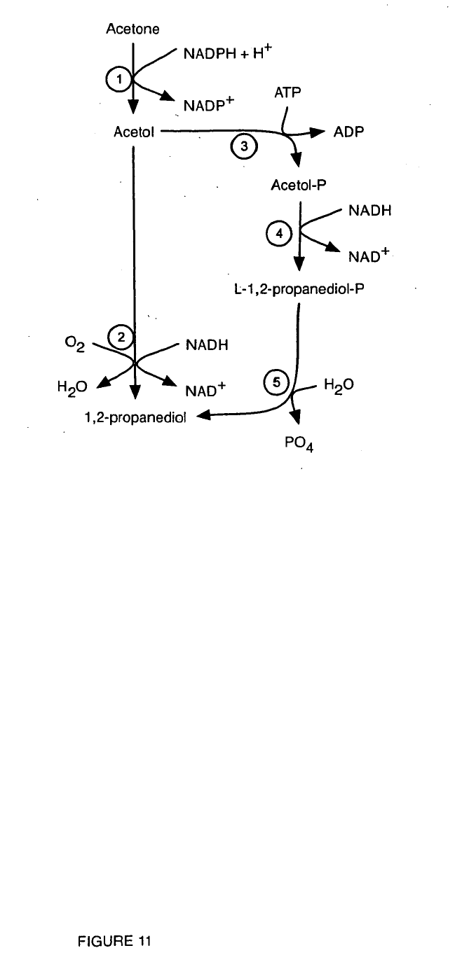

Alternatively, the gene encoding the glycerol dehydrogenase activity may be replaced with heterologous genes encoding an acetol kinase, an L-1,2-propanediol-1-phosphate dehydrogenase, and a glycerol-1-phosphate phosphatase. FIG. 11 shows two alternate pathways for the conversion of acetone to 1,2-propanediol.

Microorganisms for converting fatty acids to butyrate. In an alternate embodiment, microorganisms according to the present disclosure comprise (1) a knocked-out fadR gene; (2) a mutation in an atoC gene that provides overexpression of the microorganism's ato operon; (3) overexpression of a heterologous gene encoding an acetyl-CoA acetyltransferase or overexpression of an endogenous gene encoding an acetyl-CoA acetyltransferase; (4) a heterologous gene encoding a 3-hydroxybutyryl-CoA dehydrogenase; (5) a heterologous gene encoding a crotonase; (6) a heterologous gene encoding a butyryl-CoA dehydrogenase; and (7) a heterologous gene encoding either an acetyl-CoA:acetoacetyl-CoA transferase (E.C. 2.8.3.-2.8.3.8) or a butyrate-acetoacetate CoA transferase, or both.

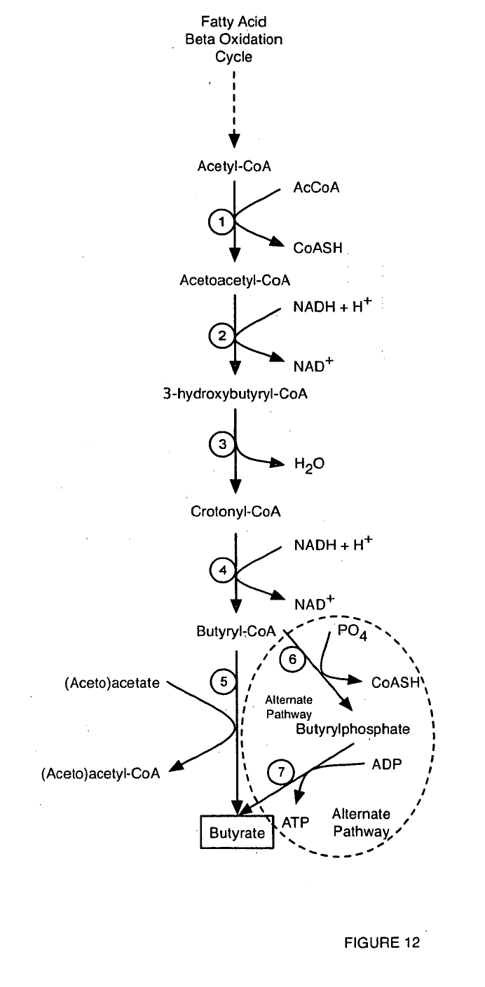

Alternatively, the heterologous gene encoding the acetate CoA ligase or butyrate-acetate CoA transferase, or both genes, may be replaced by genes encoding phosphotransbutyrylase and butyrate kinase. Phosphotransbutyrylase and butyrate kinase are described in Walter et al. (Gene 134:107-111, 1993), incorporated herein in its entirety by reference. The products of these genes work sequentially to convert butyryl-CoA to butyrylphosphate and then to butyrate. FIG. 12 shows a pathway for the conversion of fatty acids into butyrate and Tables 5 and 6 provide exemplary enzymes for the steps in the pathway.

TABLE 6

| | |
|---|---|
| E.C. No. | Exemplary Enzyme(s) |

3-hydroxybutyryl-CoA dehydrogenase

| | |
|---|---|
| 1.1.1.35 | Hbd, C. acetobutylicum (Entrez GeneID: 1118891, |
| | GenBank NP_349314) |

Crotonase (3-hydroxybutyryl-CoA dehydratase)

| | |
|---|---|
| 4.2.1.55 | Crotonase, C. acetobutylicum (Entrez GeneID: 1118895, |
| | GenBank NP_349318) |

Butyryl-CoA dehydrogenase

| | |
|---|---|
| 1.3.99.2 | Bcd, C. acetobutylicum (Entrez GeneID: 1118894, |
| | GenBank NP_349317) |

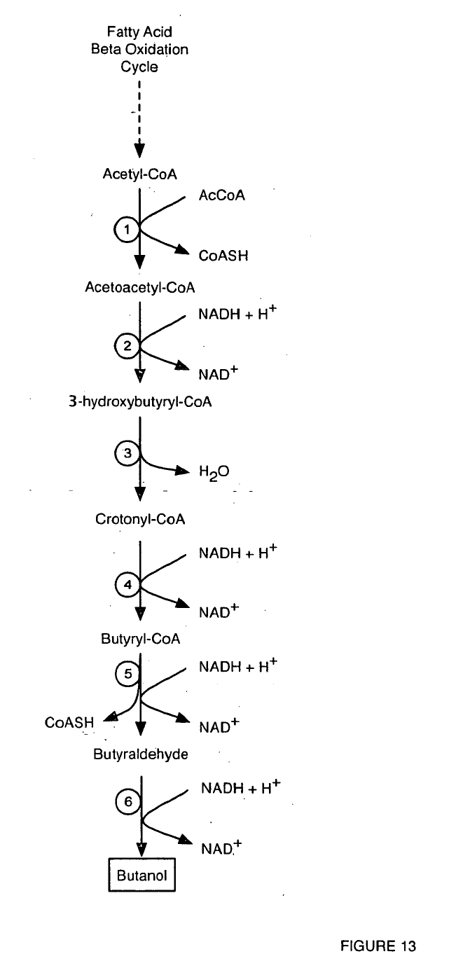

Microorganisms for converting fatty acids to butanol. In an alternate embodiment, microorganisms according to the present disclosure comprise (1) a knocked-out fadR gene; (2) a mutation in an atoC gene that provides overexpression of the microorganism's ato operon; (3) overexpression of a heterologous gene encoding an acetyl-CoA acetyltransferase or overexpression of an endogenous gene encoding an acetyl-CoA acetyltransferase; (4) a heterologous gene encoding a 3-hydroxybutyryl-CoA dehydrogenase; (5) a heterologous gene encoding a crotonase; (6) a heterologous gene encoding a butyryl-CoA dehydrogenase; (7) a heterologous gene encoding a butyraldehyde dehydrogenase; and (8) a heterologous gene encoding a butanol dehydrogenase or secondary alcohol dehydrogenase capable of converting butyraldehyde to butanol. Optionally, the microorganism may be further modified by reduction or elimination of the NADH dehydrogenase activity. Such a mutation would ensure that all NADH generated via β-oxidation is available for use in the synthesis of butanol. FIG. 13 shows a pathway for the conversion of fatty acids into butanol and Tables 5, 6, and 7 provide exemplary enzymes for the steps in the pathway.

TABLE 7

| | |
|---|---|
| E.C. No. | Exemplary Enzyme(s) |

Butyraldehyde dehydrogenase

| | |
|---|---|
| 1.2.1.57 | Bydh, C. acetobutylicum |

Butanol dehydrogenase

| | |
|---|---|
| Unassigned | Bdh, C. acetobutylicum (Entrez GeneID: 1119481 and |
| | 1119480, GenBank NP_349892 and NP_349891) |

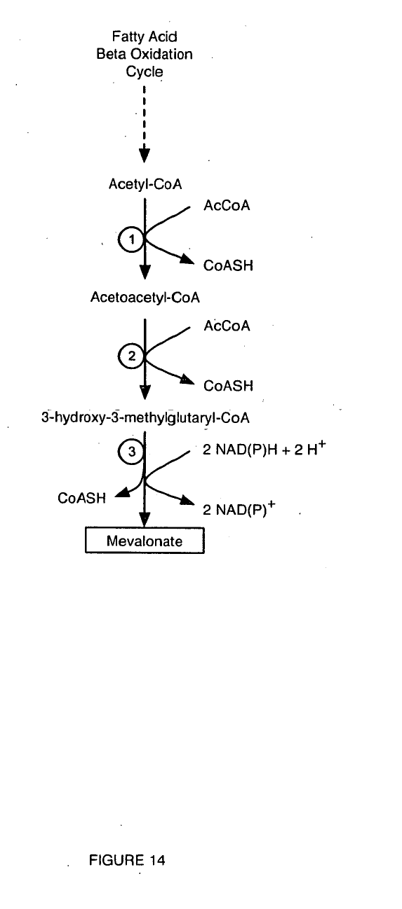

Microorganisms for converting fatty acids to mevalonate. In an alternate embodiment, microorganisms according to the present disclosure comprise (1) a knocked-out fadR gene; (2) a mutation in an atoC gene that provides overexpression of the microorganism's ato operon; (3) overexpression of a heterologous gene encoding an acetyl-CoA acetyltransferase or overexpression of an endogenous gene encoding an acetyl-CoA acetyltransferase; (4) a heterologous gene encoding a 3-hydroxy-3-methylglutaryl-CoA synthase; and (5) a heterologous gene encoding a 3-hydroxy-3-methylglutaryl-CoA reductase. FIG. 14 shows a pathway for the conversion of fatty acids to mevalonate and Tables 5 and 8 provide exemplary enzymes for the steps in the pathway.

TABLE 8

| | |
|---|---|
| E.C. No. | Exemplary Enzyme(s) |

3-hydroxy-3-methylglutaryl-CoA synthase

| | |
|---|---|
| 2.3.3.10 | Erg13, Saccharomyces cerevisiae (Entrez GeneID: 854913, |
| | GenBank NP_013580) |
| | Mva1, A. thaliana (Entrez GeneID: 826788, GenBank |
| | NP_849361 or NP_192919) |

3-hydroxy-3-methylglutaryl-CoA reductase

| | |
|---|---|
| 1.1.1.88 | HMG1 (Entrez GeneID: 854900, GenBank NP_013636) |
| | or HMG2 (Entrez GeneID: 851171, GenBank NP_13555), |
| | S. cerevisiae |
| | HMG-CoA reductase, Lactobacillus reuteri (Entrez |
| | GeneID: 5188737, GenBank YP_001270813) |

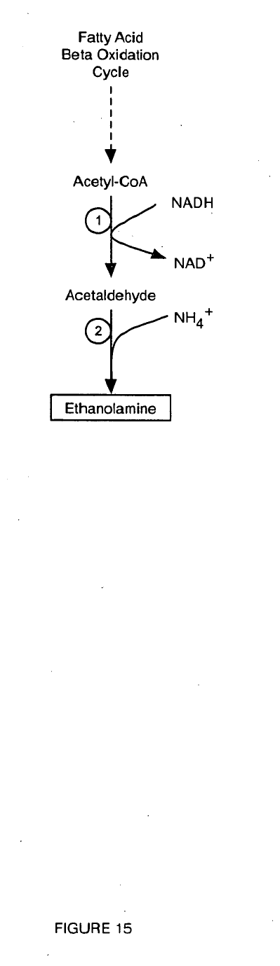

Microorganisms for converting fatty acids to ethanolamine. In an alternate embodiment, microorganisms according to the present disclosure comprise (1) a knocked-out fadR gene; (2) a mutation in an atoC gene that provides overexpression of the microorganism's ato operon; (3) overexpression of a gene encoding an endogenous acetaldehyde dehydrogenase, for example mhpF (acetaldehyde dehydrogenase 2 of E. coli, Ecocyc Gene ID M014; Entrez GeneID: 945008) or a heterologous gene encoding an acetaldehyde dehydrogenase (E.C. 1.2.1.10); and (4) genes encoding the regulatory and catalytic subunits of ethanolamine ammonia-lyase (E.C. 4.3.1.7), eutB and eutC (Ecocyc Gene ID EG50006 and EG50007; Entrez GeneID: 946924 and 946925). Such a microbial strain may require an external source of ammonia to accumulate significant quantities of ethanolamine. FIG. 15 shows a pathway for the conversion of fatty acids to ethanolamine.

Elimination of co-products. In any of the above embodiments, it may be desirable to reduce or eliminate the accumulation of unwanted co-products. For example, in many of the above embodiments, it may be necessary to reduce or eliminate the accumulation of acetate or ethanol to maximize the production of the desired chemical. Acetate production may be reduced or eliminated by, for example, a knock-out of poxB, the ackA-pta operon, or the ackA or pta gene individually or together. Ethanol may be reduced or eliminated, for example, by the deletion of the adhE gene.

Methods for converting fatty acids to desired chemicals. Embodiments of the disclosure also include methods of producing a desired product using a microorganism. The microorganisms that may be used include, but are not limited to, Escherichia coli, Lactobacillus spp., Lactococcus spp., Bacillus spp., Paenibacillus spp., Klebsiella spp., Citrobacter spp., Clostridium spp., Saccharomyces spp., Pichia spp., Zymomonas mobilis, Schizosaccharomyces pombe, and other suitable microorganisms. The desired product may be a high value chemical, such as ethanol, succinate, γ-butyrolactone, 1,4-butanediol, acetone, isopropanol, methyl acetate, 1,2-propanediol, butanol, butyrate, propionate, or ethanolamine. The microorganism may have any of the genetic modifications described above, or any combination of the various genetic modifications described in any of disclosed embodiments.

The methods generally comprise providing a medium comprising free fatty acids, monoglycerides, diglycerides, triglycerides, phospholipids, or a combination thereof and culturing the microorganism in the medium under conditions such that the microorganism converts the free fatty acids, monoglycerides, diglycerides, triglycerides, phospholipids, or combination thereof into the desired product.

The free fatty acids, monoglycerides, diglycerides, triglycerides, phospholipids, or combination thereof may be provided at 0.1% (i.e., g/l) to about 10% (i.e., 100 g/l), such as between about from about 2% and about 5% (20 g/l and 50 g/l, respectively). However, greater amounts may be used. The fatty acids, monoglycerides, diglycerides, triglycerides, phospholipids or combinations thereof, may be provided in a single dose, an initial dose that is supplemented periodically, or in an initial dose that is supplemented continuously. For example, fatty acids, monoglycerides, diglycerides, triglycerides, phospholipids, or combinations thereof, may be provided at 0.1% (i.e., 1 g/l) to about 5% (i.e., 50 g/l), such as 20 g/l, as an initial dose, and then supplemented at a rate sufficient to keep the total concentration of the fatty acids, monoglycerides, diglycerides, triglycerides, or combinations thereof at about 0.1% to about 5% as the fatty acids, monoglycerides, diglycerides, triglycerides, or combinations thereof are consumed by the microorganisms. Such concentrations may be readily determined by one of skill in the art.

The medium may also include a source of supplementary nutrients provided either as a minimal salts solution as exemplified by M-9 culture medium, or a complex medium as exemplified by Luria-Bertani broth, as described in “Molecular Cloning: a Laboratory Manual” (Maniatis, et al., eds., Cold Spring Harbor Laboratory Press, Cold Spring Harbor, N.Y., 1982). Other culture media may also be suitable. In addition, the culture medium may be supplemented with additional nutritional requirements to account for nutritional auxotrophies of the cultured microorganism, which may be readily determined by one of skill in the art.

The microorganisms are cultured in the medium containing the free fatty acids, monoglycerides, diglycerides, triglycerides, phospholipids, or a combination thereof in the presence of sufficient electron acceptors to allow the process of β-oxidation to proceed. The electron acceptors may be provided, for example, by oxygen through aeration of the culture medium, by shaking, sparging with room air or oxygen, or other appropriate methods. Alternatively, the electron acceptors may be provided by nitrate or nitrite salts, or their equivalents, added directly to the culture medium.

The microorganisms are cultured in the medium containing the free fatty acids, monoglycerides, diglycerides, triglycerides, phospholipids or a combination thereof at an appropriate temperature that is specific to each microorganism. By way of example, most E. coli strains grow best in the range of 35° C. to 39° C. Some Bacillus species grow optimally in the range of 40° C. to 45° C., and even as high as 55° C. Saccharomyces cerevisiae grows well at 28° C. to 32° C.

The high value chemicals can be isolated from the culture medium using a variety of methods that are well-known to those of skill in the art of industrial fermentation. Methods of isolating the high value chemicals include, but are not limited to, distillation, pervaporation, liquid-liquid extraction, ion exchange, crystallization, precipitation, or vacuum distillation.

While the foregoing is directed to embodiments of the present invention, other and further embodiments of the invention may be devised without departing from the basic scope thereof, and the scope thereof is determined by the claims that follow.

1.-138. (canceled)

139. A microorganism comprising:
a) i) reduced fadR expression or function, or increased expression of the fad regulon;
ii) increased expression of the atoDAEB genes; and
iii) a nucleic acid sequence encoding an alcohol dehydrogenase protein that is active under aerobic conditions;
b) i) reduced fadR expression or function, or increased expression of the fad regulon;
ii) increased expression of the atoDAEB genes; and
(1) increased expression of an acetate kinase gene or a phosphate acetyltransferase gene;
(2) increased expression of an ADP-forming acetyl-CoA synthetase activity; or
(3) expression of an acetyl-CoA synthetase activity that functions primarily to produce acetate from acetyl-CoA;
c) i) reduced fadR expression or function, or increased expression of the fad regulon;
ii) increased expression of the atoDAEB genes;
iii) reduced sucA or sucB expression or function; and
iv) reduced sdhA or sdhB expression or function;
d) i) reduced fadR expression or function, or increased expression of the fad regulon;
ii) increased expression of the atoDAEB genes;
iii) reduced sucA or sucB expression or function;
iv) reduced sdhA or sdhB expression or function;
v) increased expression or function of one or more of succinic semialdehyde dehydrogenase, succinyl-CoA:CoA transferase, or succinate-CoA ligase;
vi) increased gamma-hydroxybutyric acid dehydrogenase expression or function; and
vii) increased lactonase expression or function;
e) 1) reduced fadR expression or function, or increased expression of the fad regulon;
ii) increased expression of the atoDAEB genes;
iii) reduced sucA or sucB expression or function;
iv) reduced sdhA or sdhB expression or function;
v) increased expression or function of one or more of succinic semialdehyde dehydrogenase, succinyl-CoA:CoA transferase, or succinate-CoA ligase;
vi) increased gamma-hydroxybutyric acid dehydrogenase expression or function;
vii) increased aldehyde dehydrogenase expression or function; and
viii) increased alcohol dehydrogenase expression or function;
f) i) reduced fadR expression or function, or increased expression of the fad regulon;
ii) increased expression of the atoDAEB genes;
iii) reduced sucA or sucB expression or function;
iv) reduced sdhA or sdhB expression or function;
v) increased methylmalonyl-CoA mutase expression or function;
vi) increased methylmalonyl-CoA decarboxylase expression or function; and
vii) increased propionyl-CoA:succinate CoA transferase expression or function;
g) i) reduced fadR expression or function, or increased expression of the fad regulon;
ii) increased expression of the atoDAEB genes;
iii) increased acetyl-CoA acetyltransferase expression or function;
iv) increased acetoacetyl-CoA transferase expression or function; and
v) increased acetoacetate decarboxylase expression or function;
h) i) reduced fadR expression or function, or increased expression of the fad regulon;
ii) increased expression of the atoDAEB genes;
iii) increased acetyl-CoA acetyltransferase expression or function;
iv) increased acetoacetyl-CoA transferase expression or function;
v) increased acetoacetate decarboxylase expression or function; and
vi) a gene encoding an acetone monooxygenase;
i) i) reduced fadR expression or function, or increased expression of the fad regulon;
ii) increased expression of the atoDAEB genes;
iii) increased acetyl-CoA acetyltransferase expression or function;
iv) increased acetoacetyl-CoA transferase expression or function;
v) increased acetoacetate decarboxylase expression or function; and
vi) a gene encoding secondary alcohol dehydrogenase.
j) i) reduced fadR expression or function, or increased expression of the fad regulon;
ii) increased expression of the atoDAEB genes;
iii) increased acetyl-CoA acetyltransferase expression or function;
iv) increased acetoacetyl-CoA transferase expression or function;
v) increased acetoacetate decarboxylase expression or function; and
(1) a gene encoding an acetol monooxygenase and a gene encoding a glycerol dehydrogenase; or
(2) a gene encoding an acetol monooxygenase; a gene encoding an acetol kinase; a gene encoding an L-1,2-propanediol-1-phosphate dehydrogenase; and, a gene encoding a glycerol-1-phosphate phosphatase;
k) i) reduced fadR expression or function, or increased expression of the fad regulon;
ii) increased expression of the atoDAEB genes;
iii) increased acetyl-CoA acetyltransferase expression or function;
iv) increased 3-hydroxybutyryl-CoA dehydrogenase expression or function;
v) increased crotonase expression or function;
vi) a gene encoding butyryl-CoA dehydrogenase; and:
(1) increased acetyl-CoA:acetoacetyl-CoA transferase or butyrate-acetoacetate CoA transferase expression or function; or
(2) a gene encoding phosphotransbutyrylase and a gene encoding butyrate kinase;
l) i) reduced fadR expression or function, or increased expression of the fad regulon;
ii) increased expression of the atoDAEB genes;
iii) increased acetyl-CoA acetyltransferase expression or function;
iv) increased 3-hydroxybutyryl-CoA dehydrogenase expression or function;
v) increased crotonase expression or function;
vi) a gene encoding butyryl-CoA dehydrogenase;
vii) a gene encoding butyraldehyde dehydrogenase; and
viii) a gene encoding butanol dehydrogenase or secondary alcohol dehydrogenase;
m) i) reduced fadR expression or function, or increased expression of the fad regulon;
ii) increased expression of the atoDAEB genes;
iii) increased acetyl-CoA acetyltransferase expression or function;
iv) a gene encoding 3-hydroxy-3-methylglutaryl-CoA synthase; and
v) a gene encoding 3-hydroxy-3-methylglutaryl-CoA reductase; or
n) i) reduced fadR expression or function, or increased expression of the fad regulon;
ii) increased expression of the atoDAEB genes;
iii) increased acetaldehyde dehydrogenase expression or function; and
iv) genes encoding the regulatory and catalytic subunits of ethanolamine ammonia-lyase.

140. The microorganism of claim 139, wherein said microorganism is a bacterium.

141. The microorganism of claim 140, wherein said microorganism is a member of the genus Escherichia, Lactobacillus, Lactococcus, Bacillus, Paenibacillus, Klebsiella, Citrobacter, Clostridium, or Zymomonas.

142. The microorganism of claim 139, wherein said microorganism is a fungus.

143. The microorganism of claim 142, wherein said microorganism is a member of the genus Saccharomyces, Pichia, Schizosaccharomyces, Aspergillus, or Neurospora.

144. The microorganism of claim 139, wherein said microorganism comprised reduced fadR expression.

145. The microorganism of claim 139, wherein said microorganism comprises reduced fadR function.

146. The microorganism of claim 139, wherein said microorganism comprises increased expression of the fad regulon.

147. The microorganism of claim 139, wherein said microorganism comprises increased expression of the ato operon.

148. A method of converting free fatty acids to ethanol, acetate, succinate, gamma-butyrolactone, 1,4 butanediol, propionate, acetone, methyl acetate, isopropanol, 1,2 propanediol, butyrate, butanol, mevalonate, or ethanolamine, comprising culturing a microorganism comprising:
a) i) reduced fadR expression or function, or increased expression of the fad regulon;
ii) increased expression of the atoDAEB genes; and
iii) a nucleic acid sequence encoding an alcohol dehydrogenase protein that is active under aerobic conditions,
in a culture medium comprising free fatty acids, monoglycerides, diglycerides, triglycerides, phospholipids, or a combination thereof, thereby converting said free fatty acids to ethanol;
b) i) reduced fadR expression or function, or increased expression of the fad regulon;
ii) increased expression of the atoDAEB genes; and
(1) increased expression of an acetate kinase gene or a phosphate acetyltransferase gene;
(2) increased expression of an ADP-forming acetyl-CoA synthetase activity; or
(3) expression of an acetyl-CoA synthetase activity that functions primarily to produce acetate from acetyl-CoA,
in a culture medium comprising free fatty acids, monoglycerides, diglycerides, triglycerides, phospholipids, or a combination thereof, thereby converting said free fatty acids to acetate;
c) i) reduced fadR expression or function, or increased expression of the fad regulon;
ii) increased expression of the atoDAEB genes;
iii) reduced sucA or sucB expression or function; and
iv) reduced sdhA or sdhB expression or function,
in a culture medium comprising free fatty acids, monoglycerides, diglycerides, triglycerides, phospholipids, or a combination thereof, thereby converting said free fatty acids to succinate;
d) i) reduced fadR expression or function, or increased expression of the fad regulon;
ii) increased expression of the atoDAEB genes;
iii) reduced sucA or sucB expression or function;
iv) reduced sdhA or sdhB expression or function;
v) increased expression or function of one or more of succinic semialdehyde dehydrogenase, succinyl-CoA:CoA transferase, or succinate-CoA ligase;
vi) increased gamma-hydroxybutyric acid dehydrogenase expression or function; and
vii) increased lactonase expression or function,
in a culture medium comprising free fatty acids, monoglycerides, diglycerides, triglycerides, phospholipids, or a combination thereof, thereby converting said free fatty acids to gamma-butyrolactone;
e) i) reduced fadR expression or function, or increased expression of the fad regulon;
ii) increased expression of the atoDAEB genes;
iii) reduced sucA or sucB expression or function;
iv) reduced sdhA or sdhB expression or function;
v) increased expression or function of one or more of succinic semialdehyde dehydrogenase, succinyl-CoA:CoA transferase, or succinate-CoA ligase;
vi) increased gamma-hydroxybutyric acid dehydrogenase expression or function;
vii) increased aldehyde dehydrogenase expression or function; and
viii) increased alcohol dehydrogenase expression or function,
in a culture medium comprising free fatty acids, monoglycerides, diglycerides, triglycerides, phospholipids, or a combination thereof, thereby converting said free fatty acids to 1,4-butanediol;
f) i) reduced fadR expression or function, or increased expression of the fad regulon;
ii) increased expression of the atoDAEB genes;
iii) reduced sucA or sucB expression or function;
iv) reduced sdhA or sdhB expression or function;
v) increased methylmalonyl-CoA mutase expression or function;
vi) increased methylmalonyl-CoA decarboxylase expression or function; and
vii) increased propionyl-CoA:succinate CoA transferase expression or function,
in a culture medium comprising free fatty acids, monoglycerides, diglycerides, triglycerides, phospholipids, or a combination thereof, thereby converting said free fatty acids to propionate;
g) i) reduced fadR expression or function, or increased expression of the fad regulon;
ii) increased expression of the atoDAEB genes;
iii) increased acetyl-CoA acetyltransferase expression or function;
iv) increased acetoacetyl-CoA transferase expression or function; and
v) increased acetoacetate decarboxylase expression or function,
in a culture medium comprising free fatty acids, monoglycerides, diglycerides, triglycerides, phospholipids, or a combination thereof, thereby converting said free fatty acids to acetone;
h) i) reduced fadR expression or function, or increased expression of the fad regulon;
ii) increased expression of the atoDAEB genes;
iii) increased acetyl-CoA acetyltransferase expression or function;
iv) increased acetoacetyl-CoA transferase expression or function;
v) increased acetoacetate decarboxylase expression or function; and
vi) a gene encoding an acetone monooxygenase,
in a culture medium comprising free fatty acids, monoglycerides, diglycerides, triglycerides, phospholipids, or a combination thereof, thereby converting said free fatty acids to methyl acetate;
i) i) reduced fadR expression or function, or increased expression of the fad regulon;
ii) increased expression of the atoDAEB genes;
iii) increased acetyl-CoA acetyltransferase expression or function;
iv) increased acetoacetyl-CoA transferase expression or function;
v) increased acetoacetate decarboxylase expression or function; and
vi) a gene encoding a secondary alcohol dehydrogenase,
in a culture medium comprising free fatty acids, monoglycerides, diglycerides, triglycerides, phospholipids, or a combination thereof, thereby converting said free fatty acids to isopropanol;
j) i) reduced fadR expression or function, or increased expression of the fad regulon;
ii) increased expression of the atoDAEB genes;
iii) increased acetyl-CoA acetyltransferase expression or function;
iv) increased acetoacetyl-CoA transferase expression or function;
v) increased acetoacetate decarboxylase expression or function; and
(1) a gene encoding an acetol monooxygenase and a gene encoding a glycerol dehydrogenase; or
(2) a gene encoding an acetol monooxygenase; a gene encoding an acetol kinase; a gene encoding an L-1,2-propanediol-1-phosphate dehydrogenase; and, a gene encoding a glycerol-1-phosphate phosphatase,
in a culture medium comprising free fatty acids, monoglycerides, diglycerides, triglycerides, phospholipids, or a combination thereof, thereby converting said free fatty acids to 1,2-propanediol;
k) i) reduced fadR expression or function, or increased expression of the fad regulon;
ii) increased expression of the atoDAEB genes;
iii) increased acetyl-CoA acetyltransferase expression or function;
iv) increased 3-hydroxybutyryl-CoA dehydrogenase expression or function;
v) increased crotonase expression or function;
vi) a gene encoding butyryl-CoA dehydrogenase; and:
(1) increased acetyl-CoA:acetoacetyl-CoA transferase or butyrate-acetoacetate CoA transferase expression or function; or
(2) a gene encoding phosphotransbutyrylase and a gene encoding butyrate kinase,
in a culture medium comprising free fatty acids, monoglycerides, diglycerides, triglycerides, phospholipids, or a combination thereof, thereby converting said free fatty acids to butyrate;
l) i) reduced fadR expression or function, or increased expression of the fad regulon;
ii) increased expression of the atoDAEB genes;
iii) increased acetyl-CoA acetyltransferase expression or function;
iv) increased 3-hydroxybutyryl-CoA dehydrogenase expression or function;
v) increased crotonase expression or function;
vi) a gene encoding butyryl-CoA dehydrogenase;
vii) a gene encoding butyraldehyde dehydrogenase; and
viii) a gene encoding butanol dehydrogenase or secondary alcohol dehydrogenase,
in a culture medium comprising free fatty acids, monoglycerides, diglycerides, triglycerides, phospholipids, or a combination thereof, thereby converting said free fatty acids to butanol;
m) i) reduced fadR expression or function, or increased expression of the fad regulon;
ii) increased expression of the atoDAEB genes;
iii) increased acetyl-CoA acetyltransferase expression or function;
iv) a gene encoding 3-hydroxy-3-methylglutaryl-CoA synthase; and
v) a gene encoding 3-hydroxy-3-methylglutaryl-CoA reductase,
in a culture medium comprising free fatty acids, monoglycerides, diglycerides, triglycerides, phospholipids, or a combination thereof, thereby converting said free fatty acids to mevalonate; or
n) i) reduced fadR expression or function, or increased expression of the fad regulon;
ii) increased expression of the atoDAEB genes;
iii) increased acetaldehyde dehydrogenase expression or function; and
iv) genes encoding the regulatory and catalytic subunits of ethanolamine ammonia-lyase,
in a culture medium comprising free fatty acids, monoglycerides, diglycerides, triglycerides, phospholipids, or a combination thereof, thereby converting said free fatty acids to ethanolamine.

149. A method of producing ethanol, acetate, succinate, gamma-butyrolactone, 1,4 butanediol, propionate, acetone, methyl acetate, isopropanol, 1,2 propanediol, butyrate, butanol, mevalonate, or ethanolamine, comprising culturing a microorganism comprising:
a) i) reduced fadR expression or function, or increased expression of the fad regulon;
ii) increased expression of the atoDAEB genes; and
iii) a nucleic acid sequence encoding an alcohol dehydrogenase protein that is active under aerobic conditions,
in a culture medium comprising free fatty acids, monoglycerides, diglycerides, triglycerides, phospholipids, or a combination thereof, thereby producing ethanol;
b) i) reduced fadR expression or function, or increased expression of the fad regulon;
ii) increased expression of the atoDAEB genes; and
(1) increased expression of an acetate kinase gene or a phosphate acetyltransferase gene;
(2) increased expression of an ADP-forming acetyl-CoA synthetase activity; or
(3) expression of an acetyl-CoA synthetase activity that functions primarily to produce acetate from acetyl-CoA,
in a culture medium comprising free fatty acids, monoglycerides, diglycerides, triglycerides, phospholipids, or a combination thereof, thereby producing acetate;
c) i) reduced fadR expression or function, or increased expression of the fad regulon;
ii) increased expression of the atoDAEB genes;
iii) reduced sucA or sucB expression or function; and
iv) reduced sdhA or sdhB expression or function,
in a culture medium comprising free fatty acids, monoglycerides, diglycerides, triglycerides, phospholipids, or a combination thereof, thereby producing succinate;
d) i) reduced fadR expression or function, or increased expression of the fad regulon;
ii) increased expression of the atoDAEB genes;
iii) reduced sucA or sucB expression or function;
iv) reduced sdhA or sdhB expression or function;
v) increased expression or function of one or more of succinic semialdehyde dehydrogenase, succinyl-CoA:CoA transferase, or succinate-CoA ligase;
vi) increased gamma-hydroxybutyric acid dehydrogenase expression or function; and
vii) increased lactonase expression or function,
in a culture medium comprising free fatty acids, monoglycerides, diglycerides, triglycerides, phospholipids, or a combination thereof, thereby producing gamma-butyrolactone;
e) i) reduced fadR expression or function, or increased expression of the fad regulon;
ii) increased expression of the atoDAEB genes;
iii) reduced sucA or sucB expression or function;
iv) reduced sdhA or sdhB expression or function;
v) increased expression or function of one or more of succinic semialdehyde dehydrogenase, succinyl-CoA:CoA transferase, or succinate-CoA ligase;
vi) increased gamma-hydroxybutyric acid dehydrogenase expression or function;
vii) increased aldehyde dehydrogenase expression or function; and
viii) increased alcohol dehydrogenase expression or function,
in a culture medium comprising free fatty acids, monoglycerides, diglycerides, triglycerides, phospholipids, or a combination thereof, thereby producing 1,4-butanediol;
f) i) reduced fadR expression or function, or increased expression of the fad regulon;
ii) increased expression of the atoDAEB genes;
iii) reduced sucA or sucB expression or function;
iv) reduced sdhA or sdhB expression or function;
v) increased methylmalonyl-CoA mutase expression or function;
vi) increased methylmalonyl-CoA decarboxylase expression or function; and
vii) increased propionyl-CoA:succinate CoA transferase expression or function,
in a culture medium comprising free fatty acids, monoglycerides, diglycerides, triglycerides, phospholipids, or a combination thereof, thereby producing propionate;
g) i) reduced fadR expression or function, or increased expression of the fad regulon;
ii) increased expression of the atoDAEB genes;
iii) increased acetyl-CoA acetyltransferase expression or function;
iv) increased acetoacetyl-CoA transferase expression or function; and
v) increased acetoacetate decarboxylase expression or function,
in a culture medium comprising free fatty acids, monoglycerides, diglycerides, triglycerides, phospholipids, or a combination thereof, thereby producing acetone;
h) i) reduced fadR expression or function, or increased expression of the fad regulon;
ii) increased expression of the atoDAEB genes;
iii) increased acetyl-CoA acetyltransferase expression or function;
iv) increased acetoacetyl-CoA transferase expression or function;
v) increased acetoacetate decarboxylase expression or function; and
vi) a gene encoding an acetone monooxygenase,
in a culture medium comprising free fatty acids, monoglycerides, diglycerides, triglycerides, phospholipids, or a combination thereof, thereby producing methyl acetate;
i) i) reduced fadR expression or function, or increased expression of the fad regulon;
ii) increased expression of the atoDAEB genes;
iii) increased acetyl-CoA acetyltransferase expression or function;
iv) increased acetoacetyl-CoA transferase expression or function;
v) increased acetoacetate decarboxylase expression or function; and
vi) a gene encoding a secondary alcohol dehydrogenase,
in a culture medium comprising free fatty acids, monoglycerides, diglycerides, triglycerides, phospholipids, or a combination thereof, thereby producing isopropanol;
j) i) reduced fadR expression or function, or increased expression of the fad regulon;
ii) increased expression of the atoDAEB genes;
iii) increased acetyl-CoA acetyltransferase expression or function;
iv) increased acetoacetyl-CoA transferase expression or function;
v) increased acetoacetate decarboxylase expression or function; and
(1) a gene encoding an acetol monooxygenase and a gene encoding a glycerol dehydrogenase; or
(2) a gene encoding an acetol monooxygenase; a gene encoding an acetol kinase; a gene encoding an L-1,2-propanediol-1-phosphate dehydrogenase; and, a gene encoding a glycerol-1-phosphate phosphatase,
in a culture medium comprising free fatty acids, monoglycerides, diglycerides, triglycerides, phospholipids, or a combination thereof, thereby producing 1,2-propanediol;
k) i) reduced fadR expression or function, or increased expression of the fad regulon;
ii) increased expression of the atoDAEB genes;
iii) increased acetyl-CoA acetyltransferase expression or function;
iv) increased 3-hydroxybutyryl-CoA dehydrogenase expression or function;
v) increased crotonase expression or function;
vi) a gene encoding butyryl-CoA dehydrogenase; and:
(1) increased acetyl-CoA:acetoacetyl-CoA transferase or butyrate-acetoacetate CoA transferase expression or function; or
(2) a gene encoding phosphotransbutyrylase and a gene encoding butyrate kinase,
in a culture medium comprising free fatty acids, monoglycerides, diglycerides, triglycerides, phospholipids, or a combination thereof, thereby producing butyrate;
l) i) reduced fadR expression or function, or increased expression of the fad regulon;
ii) increased expression of the atoDAEB genes;
iii) increased acetyl-CoA acetyltransferase expression or function;
iv) increased 3-hydroxybutyryl-CoA dehydrogenase expression or function;
v) increased crotonase expression or function;
vi) a gene encoding butyryl-CoA dehydrogenase;
vii) a gene encoding butyraldehyde dehydrogenase; and
viii) a gene encoding butanol dehydrogenase or secondary alcohol dehydrogenase,
in a culture medium comprising free fatty acids, monoglycerides, diglycerides, triglycerides, phospholipids, or a combination thereof, thereby producing butanol;
m) i) reduced fadR expression or function, or increased expression of the fad regulon;
ii) increased expression of the atoDAEB genes;
iii) increased acetyl-CoA acetyltransferase expression or function;
iv) a gene encoding 3-hydroxy-3-methylglutaryl-CoA synthase; and
v) a gene encoding 3-hydroxy-3-methylglutaryl-CoA reductase,
in a culture medium comprising free fatty acids, monoglycerides, diglycerides, triglycerides, phospholipids, or a combination thereof, thereby producing mevalonate; or
n) i) reduced fadR expression or function, or increased expression of the fad regulon;
ii) increased expression of the atoDAEB genes;
iii) increased acetaldehyde dehydrogenase expression or function; and
iv) genes encoding the regulatory and catalytic subunits of ethanolamine ammonia-lyase,
in a culture medium comprising free fatty acids, monoglycerides, diglycerides, triglycerides, phospholipids, or a combination thereof, thereby producing ethanolamine.
